(19) United States
(12) Patent Application Publication
(10) Pub. No.: US 2003/0203370 A1
(43) Pub. Date: Oct. 30, 2003

(54) Method and system for partitioning sets of sequence groups with respect to a set of subsequence groups, useful for designing polymorphism-based typing assays

(76) Inventors: Zohar Yakhini, Zikhron Ya'acov (IL); Amir Ben-Dor, Bellevue, WA (US); Anya Tslenko, Chicago, IL (US); Jeff Sampson, San Francisco, CA (US); Bo U. Curry, Redwood City, CA (US)

Correspondence Address: AGILENT TECHNOLOGIES, INC., Legal Department, DL429, Intellectual Property Administration, P.O. Box 7599, Loveland, CO, 80537-0599, US

(21) Appl. No.: 10/136,884

(22) Filed: Apr. 30, 2002

(51) Int. Cl. C12Q 1/68; G06F 19/00; G01N 33/48; G01N 33/50

(52) U.S. Cl. 435/6; 702/20

(57) ABSTRACT

A method and system for partitioning DNA sequences into sets of sequences. Each set of sequences, or partition, can be fully analyzed in a single CMT/PEA genotyping assay or other type of genotyping assay resulting in a mass spectrum generated form cleaved mass tags or extended oligonucleotide primers. Four different methods for partitioning an initial set of DNA sequences, each containing an SNP, are disclosed. All four methods employ the assignability of bi-allenlic SNP sequence pairs to sequence pair partitions in order to build the partitions sequence-pair-by-sequence-pair. A partition is assignable when there is at least one unique mass spectrum peak generated in a CMT/PEA or analogous genotyping assay for each sequence of each sequence pair within the partition.

### TECHNICAL FIELD

The present invention relates to partitioning of sequences with respect to subsequences that may associate with the sequences in sequence-specific manners and, in particular, to a method and system for computationally designing polymorphism-based genotyping assays by partitioning a large number of single-nucleotide-polymorphism-containing sequences into sets of sequences that can be each concurrently assayed so that the total number of assays required for genotyping is equal to the number of sets of sequences, rather than to the much larger total number of polymorphism-containing sequences.

### BACKGROUND OF THE INVENTION

The present invention is related to the design of genotyping assays. These assays are used to determine the genetic identity of an organism or human being, and find wide application in biological research, diagnostics, and forensics, among other fields. Although the present invention is computational in nature, the present invention is directed to partitioning DNA sequences for use in genotyping assays, and an overview is therefore provided, in the following paragraphs, of the biochemistry and genetics to which the present invention is related.

Deoxyribonucleic acid (“DNA”) and ribonucleic acid (“RNA”) are linear polymers, each synthesized from four different types of subunit molecules. The subunit molecules for DNA include: (1) deoxy-adenosine, abbreviated “A,” a purine nucleoside; (2) deoxy-thymidine, abbreviated “T,” a pyrimidine nucleoside; (3) deoxy-cytosine, abbreviated “C,” a pyrimidine nucleoside; and (4) deoxy-guanosine, abbreviated “G,” a purine nucleoside. The subunit molecules for RNA include: (1) adenosine, abbreviated “A,” a purine nucleoside; (2) uracil, abbreviated “U,” a pyrimidine nucleoside; (3) cytosine, abbreviated “C,” a pyrimidine nucleoside; and (4) guanosine, abbreviated “G,” a purine nucleoside. FIG. 1 illustrates a short DNA polymer 100, called an oligomer, composed of the following subunits: (1) deoxy-adenosine 102; (2) deoxy-thymidine 104; (3) deoxy-cytosine 106; and (4) deoxy-guanosine 108. When phosphorylated, subunits of DNA and RNA molecules are called “nucleotides” and are linked together through phosphodiester bonds 110-115 to form DNA and RNA polymers. A linear DNA molecule, such as the oligomer shown in FIG. 1, has a 5′ end 118 and a 3′ end 120. A DNA polymer can be chemically characterized by writing, in sequence from the 5′ end to the 3′ end, the single letter abbreviations for the nucleotide subunits that together compose the DNA polymer. For example, the oligomer 100 shown in FIG. 1 can be chemically represented as “ATCG.” A DNA nucleotide comprises a purine or pyrimidine base (e.g. adenine 122 of the deoxy-adenylate nucleotide 102), a deoxy-ribose sugar (e.g. deoxy-ribose 124 of the deoxy-adenylate nucleotide 102), and a phosphate group (e.g. phosphate 126) that links one nucleotide to another nucleotide in the DNA polymer. In RNA polymers, the nucleotides contain ribose sugars rather than deoxy-ribose sugars. In ribose, a hydroxyl group takes the place of the 2′ hydrogen 128 in a DNA nucleotide. RNA polymers contain uridine nucleosides rather than the deoxy-thymidine nucleosides contained in DNA. The pyrimidine base uracil lacks a methyl group (130 in FIG. 1) contained in the pyrimidine base thymine of deoxy-thymidine.

The DNA polymers that contain the organization information for living organisms occur in the nuclei of cells in pairs, forming double-stranded DNA helixes. One polymer of the pair is laid out in a 5′ to 3′ direction, and the other polymer of the pair is laid out in a 3′ to 5′ direction. The two DNA polymers in a double-stranded DNA helix are therefore described as being anti-parallel. The two DNA polymers, or strands, within a double-stranded DNA helix are bound to each other through attractive forces including hydrophobic interactions between stacked purine and pyrimidine bases and hydrogen bonding between purine and pyrimidine bases, the attractive forces emphasized by conformational constraints of DNA polymers. Because of a number of chemical and topographic constraints, double-stranded DNA helices are most stable when deoxy-adenylate subunits of one strand hydrogen bond to deoxy-thymidylate subunits of the other strand, and deoxy-guanylate subunits of one strand hydrogen bond to corresponding deoxy-cytidilate subunits of the other strand.

FIGS. 2A-B illustrate the hydrogen bonding between the purine and pyrimidine bases of two anti-parallel DNA strands. FIG. 2A shows hydrogen bonding between adenine and thymine bases of corresponding adenosine and thymidine subunits, and FIG. 2B shows hydrogen bonding between guanine and cytosine bases of corresponding guanosine and cytosine subunits. Note that there are two hydrogen bonds 202 and 203 in the adenine/thymine base pair, and three hydrogen bonds 204-206 in the guanosine/cytosine base pair, as a result of which GC base pairs contribute greater thermodynamic stability to DNA duplexes than AT base pairs. AT and GC base pairs, illustrated in FIGS. 2A-B, are known as Watson-Crick (“WC”) base pairs.

Two DNA strands linked together by hydrogen bonds forms the familiar helix structure of a double-stranded DNA helix. FIG. 3 illustrates a short section of a DNA double helix 300 comprising a first strand 302 and a second, anti-parallel strand 304. The ribbon-like strands in FIG. 3 represent the deoxyribose and phosphate backbones of the two anti-parallel strands, with hydrogen-bonding purine and pyrimidine base pairs, such as base pair 306, interconnecting the two strands. Deoxy-guanylate subunits of one strand are generally paired with deoxy-cytidilate subunits from the other strand, and deoxy-thyrnidilate subunits in one strand are generally paired with deoxy-adenylate subunits from the other strand. However, non-WC base pairings may occur within double-stranded DNA. Generally, purine/pyrimidine non-WC base pairings contribute little to the thermodynamic stability of a DNA duplex, but generally do not destabilize a duplex otherwise stabilized by WC base pairs. However, purine/purine base pairs may destabilize DNA duplexes.

Double-stranded DNA may be denatured, or converted into single stranded DNA, by changing the ionic strength of the solution containing the double-stranded DNA or by raising the temperature of the solution. Single-stranded DNA polymers may be renatured, or converted back into DNA duplexes, by reversing the denaturing conditions, for example by lowering the temperature of the solution containing complementary single-stranded DNA polymers. During renaturing or hybridization, complementary bases of anti-parallel DNA strands form WC base pairs in a cooperative fashion, leading to regions of DNA duplex. Strictly A-T and G-C complementarity between anti-parallel polymers leads to the greatest thermodynamic stability, but partial complementarity including non-WC base pairing may also occur to produce relatively stable associations between partially-complementary polymers. In general, the longer the regions of consecutive WC base pairing between two nucleic acid polymers, the greater the stability of hybridization between the two polymers under renaturing conditions.

The DNA in living organisms occurs as extremely long double-stranded DNA polymers known as chromosomes. Each chromosome may contain millions of base pairs. The base-pair sequence in a chromosome is logically viewed as a set of long subsequences that include regulatory regions to which various biological molecules may bind, structural regions consisting of repeated short sequences, and genes. A gene generally encodes the amino-acid sequence of a protein, with base-pair triples within the exon region of a gene coding for specific amino acids within the protein.

When cells divide, the double-stranded chromosomes are replicated in a process logically equivalent to separating the two DNA strands of a chromosome and synthesizing a new, complementary strand for each of the two separated strands, resulting in two chromosomes, each containing an original strand and a newly synthesized strand. DNA synthesis is carried out by the enzyme DNA polymerase. This enzyme polymerizes nucleotide triphosphate monomers into a DNA polymer complementary to a DNA polymer that serves as a template for the DNA polymerase.

Chromosomes are transcribed in an organism by an RNA polymerase to produce messenger RNA molecules (“mRNA”) that, in turn, serve as templates for translation of the base-pair sequence of the mRNA into protein molecules. The amino-acid sequence of protein molecules is thus determined by the base-pair sequence of the messenger RNA, which is, in turn, complementary to, and determined by, the base-pair sequence within a corresponding gene.

In general, the organisms within a species commonly share the DNA sequences of the genes contained within their chromosomes. However, slight variations of gene sequences occur within the individuals of each species. These slight variations are reflected in the biochemical and physical characteristics of individuals of the species. Hair color, eye color, growth patterns, disease susceptibility, metabolism, and many other characteristics that vary among individuals of a species are attributable to variations in gene sequences. In addition, non-protein-coding regions of the genome are also shared, in some cases as conservatively or more conservatively as protein-coding regions, and, in other cases, less conservatively. Sequence differences in non-protein-coding regions between individuals may also lead to observably different traits and characteristics of the individuals. For example, genes are generally associated with DNA control sequences that provide a basis for transcriptional control of gene expression. A modified control region may as effectively lead to low concentrations or the absence of a protein function as a serious mutation in the gene encoding the protein.

Genes that vary in sequence between individuals of a specimen are known as polymorphic genes. Polymorphisms are sequence differences between individuals of a species both within genes as well as in non-protein-encoding regions of the genome. In many cases, a polymorphism may comprise a single base-pair difference within an otherwise common sequence. Thus, for example, one polymorphic form of the gene may include an A-T base pair at a particular position within the sequence while another polymorphic form of a gene contains a G-C base pair at that position. Polymorphisms that differ by a single base pair within an otherwise relatively long common sequence are known as single-nucleotide polymorphisms (“SNPs”). Although certain embodiments of the present invention are directed specifically to SNPs, the techniques of the present invention may be employed for analyzing other types of polymorphisms, including multiple sites of base-pair differences within a short stretch of DNA.

Thousands of common SNPs have been identified within the human genome. When a sufficient number of SNPs are considered, each individual can be identified by the particular SNPs within his or her genome. Thus, determining the SNPs residing within the genome of a particular individual is a powerful means for identifying that individual. Moreover, by knowing the SNPs present within the genome of an individual, it may be possible to tailor drugs and drug therapies to that individual. Identifying SNPs within individuals exhibiting a particular disease or pattern of symptoms may allow researchers to attribute the disease or pattern of symptoms to one or more gene polymorphisms, leading to development of new drugs and treatments for the disease of pattern or symptoms. During the past twenty years, a very large number of common SNPs have been identified within the human population, and sophisticated genotyping assays or, in other words, biochemical procedures for determining the SNPs present within a particular individual, have been developed. In addition, polymorphisms are invaluable for genetic linkage research, and for developing genetic associations with observable traits and characteristics of individuals of a species.

One technique currently used for SNP-based genotyping employs small oligonucleotides labeled with cleavable mass tags (“CMT”) as primers for the polymerase extension assay (“PEA”). The CMT/PEA SNP-genotyping technique is described below with reference to FIGS. 4-10.

FIG. 4 shows a diagram of six polymorphic pairs of double-stranded DNA sequences. Each double-stranded sequence is labeled with a sequence number consisting of the letter “S” followed by a number and, for half of the sequences, an apostrophe. The two double-stranded sequences of an SNP pair share the same number, with the label of one sequence of the pair ending in an apostrophe. Thus, for example, the first polymorphic pair 402 comprises double-stranded sequence S1 404 and double-stranded sequence S1′ 406. In each double-stranded sequence, one single-stranded sequence is labeled with a “+” subscript, and the other, complementary single-stranded sequence is labeled with a “−” subscript, corresponding to the two complementary sequences within a single, double-stranded DNA sequence. Thus, sequences S1₊ and S1₋ are complementary single-stranded sequences that together form a first double-stranded DNA-polymer sequence, S1, and complementary single-stranded sequences S1₊′ and S1₋′ form a second double-stranded DNA-polymer sequence, S1′, polymorphic to the first double-stranded DNA-polymer sequence. The polymorphic pair S1 and S1′ share a common double-stranded-DNA nucleotide sequence with the exception that polymorphism S1 contains an adenosine/guanosine nucleotide pair 408 at the position at which polymorphism S1′ contains a cytosine/thymidine nucleotide pair 410. S1′ and S1 together represent an SNP pair.

For the example illustrated in FIGS. 4-10, consider different genotypes with respect to polymorphisms S1, S2, S3, S4, S5, S6, S1′, S2′, S3′, S4′, S5′, and S6′. A particular individual may have, with respect to the SNPs illustrated in FIG. 4, the genotype S1/S1′, S2/S2′, S3/S3, S4/S4, S5/S5′, S6/S6, while a different individual may have the genotype S1′/S1′, S2/S2, S3/S3, S4/S4′, S5/S5, S6/S6, assuming that the sequences are contained in autosomes, or paired, homologous chromosomes. Were the sequences contained in sex chromosomes, by contrast, then only a single copy, rather than two copies, of each polymorphic double-stranded sequence would be expected in a given individual. The goal of a genotyping assay for the SNPs illustrated in FIG. 4 is to determine to which genotype an individual belongs by analyzing a solution including multiple copies of single-stranded sequences including both complementary double-stranded sequences of one or both of the six polymorphic pairs. Even for the small number of SNP pairs illustrated in FIG. 4, there are 3⁶, or 729, different possible genotypes. Thus, the example genotyping assay must distinguish a particular genotype from 729 different possible genotypes by a biochemical technique. Normally, of course, a genotype assay identifies many hundreds or thousands of different SNP sequences in order to determine the genotype of an individual. The solution of sequences that is analyzed in order to determine a genotype of an individual may be produced in a number of different ways, including polymerase chain reaction (“PCR”) amplification of small chromosome fragments generated by exposing chromosomes, isolated from a tissue sample, to restriction enzymes or other DNA cleaving agents. FIG. 5 shows certain of the DNA sequences that would occur in a sample solution generated by PCR amplification of chromosome fragments from an individual having the genotype S1/S1′, S2/S2′, S3/S3, S4/S4, S5/S5′, S6/S6.

The CMT/PEA technique employs a large number of small oligonucleotide primers complementary to subsequences within the SNP sequences that may be present in a sample solution. These small oligonucleotide primers are labeled with cleavable mass tags, chemical moieties, generally covalently linked to the oligonucleotide primers, having different molecular weights that can be distinguished by mass spectrometry. In one technique, the bond linking a cleavable mass tag to an oligonucleotide primer is photolabile, allowing the cleavable mass tag to be cleaved from the oligonucleotide primer by exposing the photolabile bond to electromagnetic radiation of a particular wavelength in the visible-light or ultraviolet-light region of the spectrum.

FIG. 6 shows a set of small oligonucleotide primers with cleavable mass tags suitable for CMT/PEA analysis of a solution containing a set of amplified sequences, such as the set of sequences shown in FIG. 5. The oligonucleotide primers in FIG. 6 are shown using a diagrammatic convention similar to that used in FIGS. 4 and 5. Each oligonucleotide primer is represented by a vertical rectangle, such as oligonucleotide primer 602. A cleavable mass tag is bound to the 5′ end of each oligonucleotide primer. The cleavable mass tag is shown, in FIG. 6, as a circle containing a number, such as cleavable mass tag 604. The number corresponds to a cleavable mass tag having a particular molecular weight. For the sake of the example, the molecular weights of the cleavable mass tags are proportional to integers within the range 1 to 30. In FIG. 6, certain of the oligonucleotide primers have sequences complementary to subsequences of the sequences, shown in FIG. 4, containing the position of the SNP. For example, oligonucleotide primer 606 is a subsequence of the “+” strand of the polymorphism S6 that represents one of the two polymorphic forms S6 and S6′, and oligonucleotide primer 608 is a subsequence of the “−” strand of the S2′ polymorphism.

In some cases, the oligonucleotide primers are generated by standard DNA-synthesis methods, while, in other cases, the oligonucleotide primers are generated by a combinatorial technique. The set of oligonucleotide primers may contain all possible oligonucleotide sequences of a particular length, or may contain a random set of oligonucleotide sequences. However, all copies of a particular oligonucleotide primer are uniformly labeled within one cleavable mass tag. Note also that the number of different cleavable mass tags is normally smaller than the number of oligonucleotide primers, so that a number of different oligonucleotide primers have the same cleavable mass tag. In the CMT/PEA genotyping technique, a solution containing a mixture of CMT-labeled oligonucleotide primers, such as the CMT-labeled oligonucleotide primers shown in FIG. 6, is introduced into a sample solution containing PCR-amplified DNA sequences, such as the DNA sequences shown in FIG. 5. Oligonucleotide primers complementary to subsequences within the DNA sequences bind relatively strongly, by Watson-Crick base pairing, to those subsequences, while oligonucleotide primers that are not base-pair complementary to subsequences within the DNA sequences remain unbound or bind weakly and generally non-specifically. FIG. 7 shows the different possible DNA-sequence/CMT-labeled oligonucleotide-primer complexes resulting from hybridization of CMT-labeled oligonucleotide primers of FIG. 6 with the DNA sequences of FIG. 5. In FIG. 7, for example, the “+” strand of the double-stranded DNA polymorphism S1 702, 704 is shown to hybridize with two different CMT-labeled oligonucleotide primers 706-708, respectively. The “−” strand of the ploymorphism S2 710-712 forms two complexes with oligonucleotide primers 714 and 716, respectively.

In a next step, a solution containing DNA polymerase and the four triphosphate nucleotides is introduced into the solution containing DNA/CMT-labeled-oligonucleotide-primer complexes. The DNA polymerase extends the hybridized primers by template-directed polymerization of the nucleotide triphosphate monomers, using the DNA sequence as a template. DNA polymerase can extend only oligonucleotide primers hybridized to DNA sequences. Oligonucleotide primers not complementary to subsequences of the DNA sequences are therefore not extended by the DNA polymerase. FIG. 8 shows the DNA/CMT-labeled-oligonucleotide-primer complexes following DNA polymerase-mediated extension.

Following extension of CMT-labeled oligonucleotide primers bound to DNA polymers, the DNA-polymer/CMT-labeled-oligonucleotide-primer complexes are denatured by subjecting the complexes to any of various denaturing conditions, such as increased temperature or addition of organic solvents. The resulting solution contains the single-stranded DNA sequences, CMT-labeled primers extended by the DNA-polymerase-mediated DNA synthesis, and CMT-labeled primers that were not complementary to subsequences within the DNA sequences and that were therefore not extended.

FIG. 9 shows the next step in the CMT/PEA technique. The solution 902 containing single-stranded DNA polymers, such as DNA polymer 904, extended CMT-labeled primers, such as extended CMT-labeled primer 906, and CMT-labeled primers not extended by the DNA-polymerase-mediated DNA synthesis, such as DNA extended CMT-labeled primer 908, is sepatated using high performance liquid chromatography (“HPLC”) or another molecular separation technique. The HPLC technique involves forcing the solution 902 through a separation column at relatively high pressure, which separates the molecules based on their relative affinities for the solution and for the solid phase column matrix. Various types of HPLC can be used to elute the different sizes of oligonucleotides in different orders. In FIG. 9, a time-ordered sequence of fractions collected from an HPLC column are shown, with the first eluted fraction 910 containing the shortest, smallest-molecular-weight oligonucleotides and the most recently eluted fraction 912 primarily containing the longer DNA oligonucleotides. The order of elution may be reversed depending on the type of HPLC column and the nature of the solution used. In the fractionation depicted in FIG. 9, fraction 914 primarily contains extended CMT-labeled oligonucleotide primers. Thus, an HPLC or other molecular separation technique is employed to produce a fraction of the original solution 902 containing extended CMT-labeled oligonucleotide primers.

Next, the purified extended CMT-labeled oligonucleotide primers are exposed to visible or UV light to cleave the mass tags from the oligonucleotides. The mass tag molecules are separated from the oligonucleotide primers to produce a solution of mass tag molecules cleaved from extended CMT-labeled oligonucleotide primers, and thus corresponding to oligonucleotide primers having sequences complementary to subsequences within the original DNA polymers prepared by PCR amplification of chromosome fragments. The cleaved mass tags are analyzed by mass spectrometry to produce a mass spectrum showing the molecular weights of the various mass tags originally borne by oligonucleotide primers complementary to subsequences of the genomic DNA polymers.

Thus, the CMT/PEA method starts with single-stranded chromosome fragments produced from a tissue sample. The chromosome fragments are exposed to mass-tag-labeled oligonucleotide primers, some of which are complementary, in sequence, to subsequences of the single-stranded chromosome fragments. Those oligonucleotide primers that are complementary to single-stranded chromosome fragments are extended, via DNA-polymerase-mediated strand elongation. The extended primers can be separated from non-extended primers to produce an extended-primer-fingerprint of subsequences within the chromosomal fragments. The fingerprint can be analyzed by cleaving the mass tags from the extended primers, and identifying the mass tags via mass spectrometry.

FIG. 10 shows a portion of a hypothetical mass spectrum corresponding to mass tags cleaved from the extended oligonucleotide primers shown in FIG. 8. The vertical axis 1002 corresponds to quantity, and the horizontal axis 1004 corresponds to molecular weight. Peaks in the mass spectrum thus show the molecular weights of the mass tags that were carried by CMT-labeled oligonucleotide primers having sequences complementary to subsequences within the genomic DNA. Of course, in an actual experiment, many different randomly generated primer sequences may be complementary to more than one DNA polymer, and will generally appear in the mass spectrum along with the oligonucleotide primers complementary to subsequences including SNP-based substitutions. Moreover, the CMT/PEA technique normally employs thousands of oligonucleotide primers, and the resulting mass spectra are quite complex. However, a small number of oligonucleotide primers, such as those shown in FIG. 7, will characteristically bind to SNP-containing subsequences, and thus serve as fingerprints for particular SNPs. The presence of the mass tags carried by the SNP-characteristic oligonucleotide primers in a mass spectrum can thus serve as an indication of the SNP present within an organism.

As discussed above, there are generally a much smaller number of mass tags than primer sequences, and many different primers may be therefore identically labeled. Thus, fingerprinting SNPs using the CMT/PEA technique involves extracting a small fingerprint signal from complex mass spectra in which the presence of a particular mass tag may indicate extension of many different oligonucleotide primers. Moreover, even considering only SNP-characteristic primers, there may be a number of different genotypes that map to a particular mass spectrum. For example, the mass spectrum shown in FIG. 10 corresponds to mass tags removed from primers, extended as shown in FIG. 8, characteristic for the genotype S1/S1′, S2/S2′, S3/S3, S4/S4, S5/S5′, S6/S6. However, the same mass spectrum is produced by the above-illustrated CMT/PEA technique when applied to the example genotype S1/S1′, S2/S2′, S3/S3′, S4′/S4′, S5/S5′, S6/S6, as can be see noting that, in FIG. 6, the characteristic primers for polymorphism S3 610-613 carry the same mass tags as the primer sequences characteristic for polymorphism S4′ 614-617 and the characteristic primers for polymorphism S3′ carry the same mass tags as the primer sequences characteristic for polymorphism S4. Therefore, the genotyping assay illustrated in FIGS. 4-10 needs to be carried out in multiple assay steps in order to distinguish genotype S1/S1′, S2/S2′, S3/S3, S4/S4, S5/S5′, S6/S6 from genotype S1/S1′, S2/S2′, S3′/S3′, S4′/S4′, S5/S5′, S6/S6. Instead of hybridizing the set of oligonucleotide primers to all chromosome fragments, including fragments produced from all six polymorphisms, the chromosome fragments need to be partitioned into sets that generate unambiguous mask-tag fingerprints under the CMT/PEA assay technique. Thus, to resolve the above-two noted genotypes, the fragments produced from the double-stranded sequences shown in FIG. 5 need to be divided into two groups, one group containing fragments from the polymorphism S3/S3′, and the other group containing fragments from the polymorphism S4/S4′. Note that the partitioning can be accomplished by partitioning PCR primers used to amplify DNA fragments.

For SNP-based genotype assays to further penetrate the lucrative diagnostics and forensics markets, the cost of SNP-based genotyping assay procedures need to be decreased as far as possible. One way to decrease costs is to increase the multiplexing of individual assays or, in other words, to decrease the number of individual assays of DNA samples needed to be performed in order to obtain genotype information during SNP-based genotyping. Each application of oligonucleotide primers to a sample solution, followed by primer extension, HPLC, and mass spectroscopy, adds an incremental cost to the entire procedure. Thus, in terms of the above-described example, the genotyping assay is considerably less expensive if all possible genotypes can be distinguished one from another in a single CMT/PEA assay, without need for partitioning the gene fragments into multiple groups. However, because a smaller range of mass tags is available than the range of primer sequences, normal genotype assays generally require partitioning. In order to minimize the cost of the genotyping assays, the partitioning needs to be as close to optimal as possible, so that the fewest possible number of CMT/PEA steps need to be carried out in order to achieve an unambiguous genotyping. This same partitioning problem also occurs in a variety of other types of SNP-based genotyping assays, including the cleavable mass-tag/ligation assay and a direct PEA/mass spectroscopy technique that relies on directly measuring the mass of the extended primers, rather than measuring the masses of cleavable tags.

Computational partitioning problems are generally time and resource intensive. Brute-force, combinatorial techniques are generally impossibly slow. Thus, designers, manufacturers, and users of SNP-based genotyping assays have recognized the need for an efficient computational technique for designing SNP-based genotyping assays in order to maximize multiplexing by minimizing the number of partitions of sample DNA fragments needed in order to achieve unambiguous genotyping.

### SUMMARY OF THE INVENTION

The present invention relates to partitioning sequences with respect to complementary subsequences, and a number of described embodiments related to computational techniques for partitioning DNA sequences into sets of DNA sequences. Each set of DNA sequences, or partition, can be fully analyzed in a single CMT/PEA genotyping assay or other type of genotyping assay that produces, as an intermediate step, a mass spectrum generated from cleaved mass tags or mass-tag-labeled oligonucleotide primers. Four different methods for partitioning an initial set of DNA sequences, each containing an SNP, are disclosed. All four methods employ the assignability of bi-allelic SNP-sequence pairs to sequence-pair partitions in order to build the partitions sequence-pair-by-sequence-pair. A partition is assignable when there is at least one unique mass spectrum peak generated in a CMT/PEA or analogous genotyping assay for each sequence of each sequence pair within the partition. Sequence pairs are added to the most favorable candidate partition selected form among currently existing partitions and, when no suitable candidate partition can be identified, are added to a newly created partition. The most suitable candidate partition is the partition that, upon adding a sequence pair, generates a larger partition that is both assignable with respect to a set of oligonucleotide primers and results in a minimal number of mass-spectrum peaks as the result of a CMT/PEA or similar genotyping assay. The four disclosed methods include two methods for designing assays that employ a single set of oligonucleotide primers, called the “minimal partition heuristic method” and the “maximal set heuristic method,” and two methods that employ different sets of primers, called the “modified minimal partition heuristic,” and the “modified maximal set heuristic.” The four disclosed methods can be easily modified for partitioning polymorphisms other than bi-allelic SNPs, including polymorphisms involving base substitutions at more than one position and tri-allelic and tetra-allelic SNPs, microsatellite polymorphisms, as well as different genotyping techniques besides CMT/PEA.

### BRIEF DESCRIPTION OF THE DRAWINGS

FIG. 1 illustrates a short DNA polymer.

FIG. 2A shows hydrogen bonding between adenine and thymine bases of corresponding adenosine and thymidine subunits.

FIG. 2B shows hydrogen bonding between guanine and cytosine bases of corresponding guanosine and cytosine subunits.

FIG. 3 illustrates a short section of a DNA double helix.

FIG. 4 shows a diagram of six polymorphic pairs of DNA sequences.

FIG. 5 shows certain of the DNA sequences that would occur in a sample solution generated by PCR amplification of chromosome fragments from an individual having the genotypes S1/S1′, S2/S2′, S3/S3, S4/S4, S5/S5′, S6/S6.

FIG. 6 shows a set of small oligonucleotide primers with cleavable mass tags suitable for CMT/PEA analysis of a solution containing a set of amplified sequences, such as the set of sequences shown in FIG. 5.

FIG. 7 shows the different possible DNA-polymer/CMT-labeled oligonucleotide-primer complexes resulting from hybridization of CMT-labeled oligonucleotide primers of FIG. 6 with the DNA sequences of FIG. 5.

FIG. 8 shows the DNA-polymer/CMT-labeled oligonucleotide-primer complexes following DNA polymerase-mediated extension.

FIG. 9 shows the next step in the CMT/PEA technique.

FIG. 10 shows a portion of a hypothetical mass spectrum corresponding to mass tags cleaved from the extended oligonucleotide primers shown in FIG. 8.

FIG. 11 is a flow-control diagram illustrating the minimal partition heuristic method described above in mathematical pseudocode.

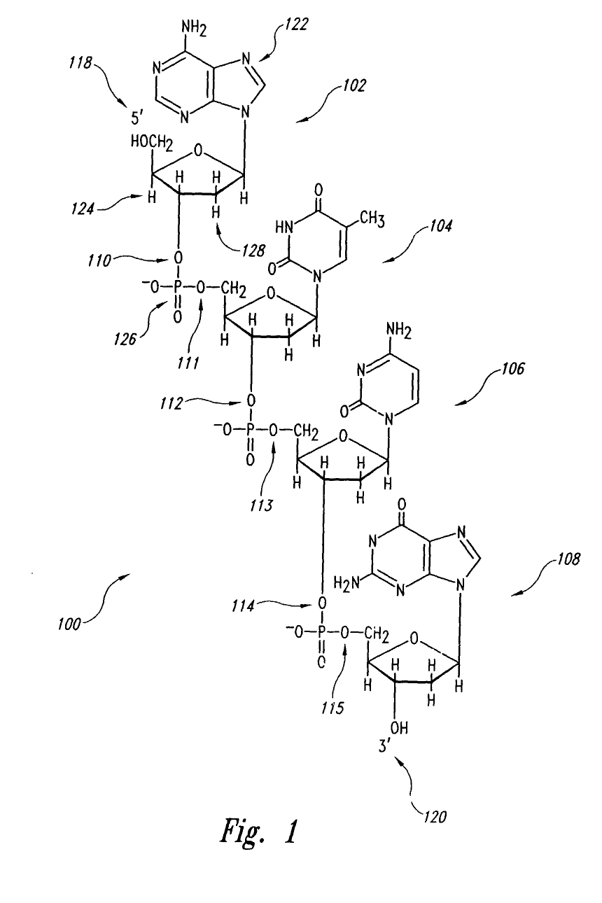

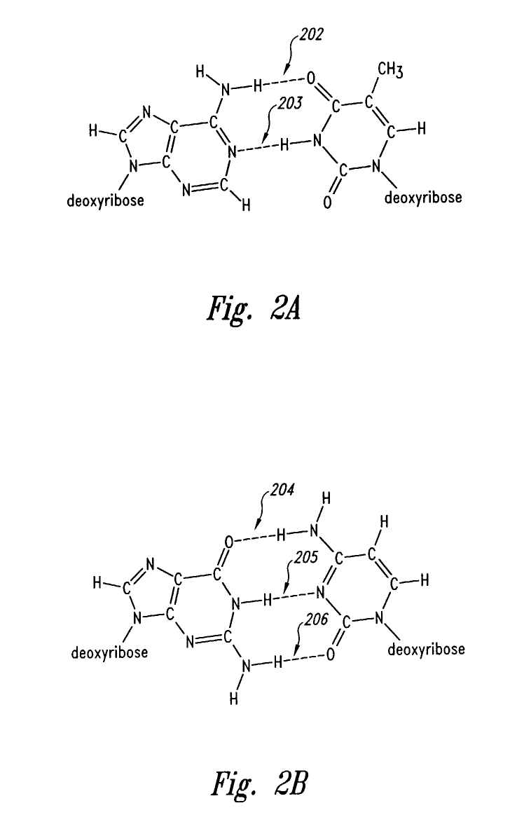

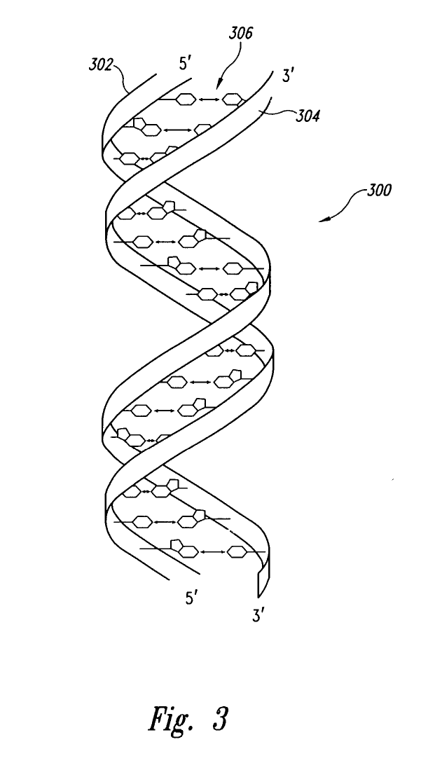

### DETAILED DESCRIPTION OF THE INVENTION

The present invention is related to partitioning DNA sequences containing SNPs into subsequences that can be concurrently genotyped using any of various SNP-based genotyping techniques, such as CMT/PEA, described above in the background. Short amplicons for each set of SNP-containing DNA sequences can be prepared from a sample solution of chromosome fragments by selectively amplifying, using PCR, the SNP-containing sequences of the set. The set of amplified SNP-containing sequences can then be exposed to a large set of short oligonucleotide primers and the remaining steps of the CMT/PEA technique can be performed to produce a mass spectrum from which SNP-characteristic masses can be extracted to unambiguously determine which of the possible alleles for each SNP are present in the organism from which the chromosome fragments were isolated. The present invention is particularly directed towards a computational method and system for optimally, or near optimally, partitioning SNP-containing sequences into sets of SNP-containing sequences, each set containing SNP-containing sequences that can be unambiguously genotyped in one CMT/PEA assay.

First, a mathematical basis for partitioning is provided. Next, a number of methods for partitioning SNP-containing sequences are described in mathematical pseudocode. Following the mathematical pseudocode, a flow-control approach to describing a method for partitioning SNP-containing sequences is provided. Finally, a C++-like implementation of a partitioning method is provided in order to demonstrate one approach to implementing the methods expressed in mathematical pseudocode in a commonly used computer language.

### Mathematical Model

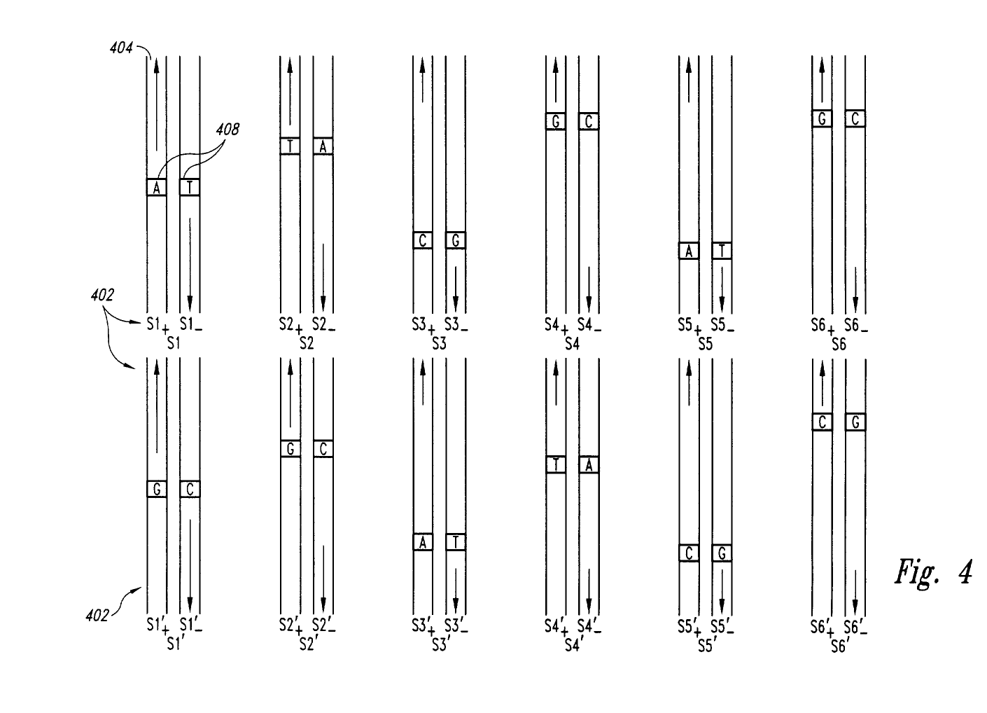

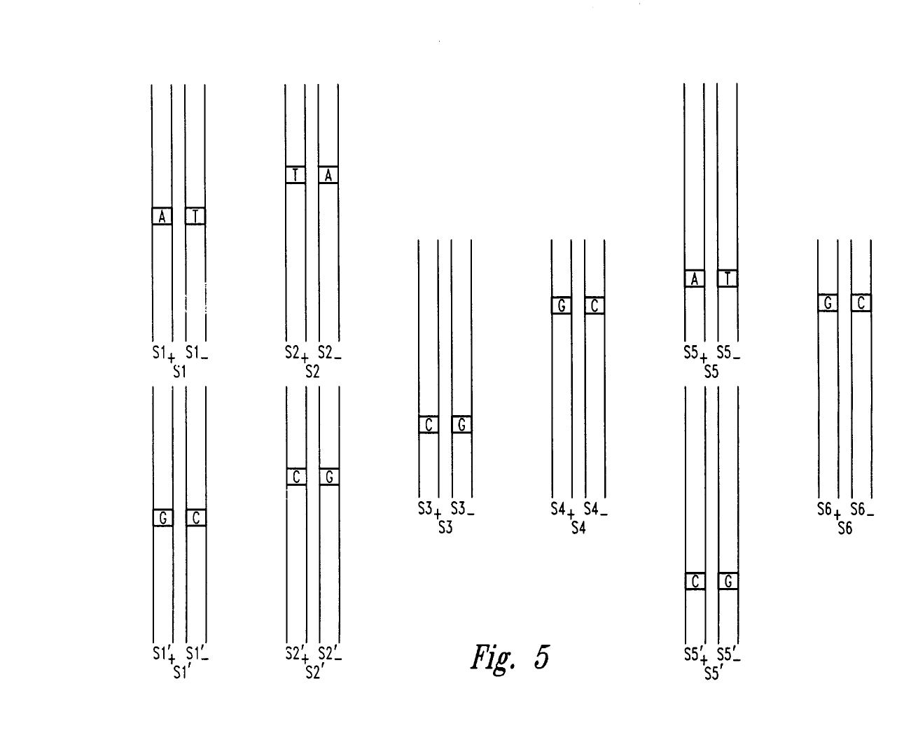

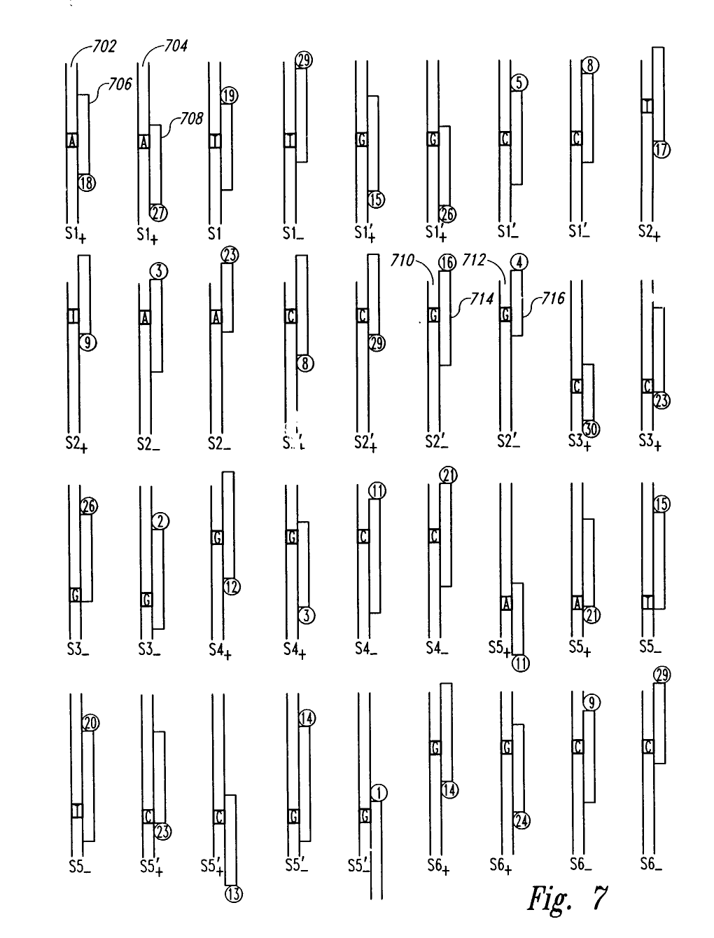

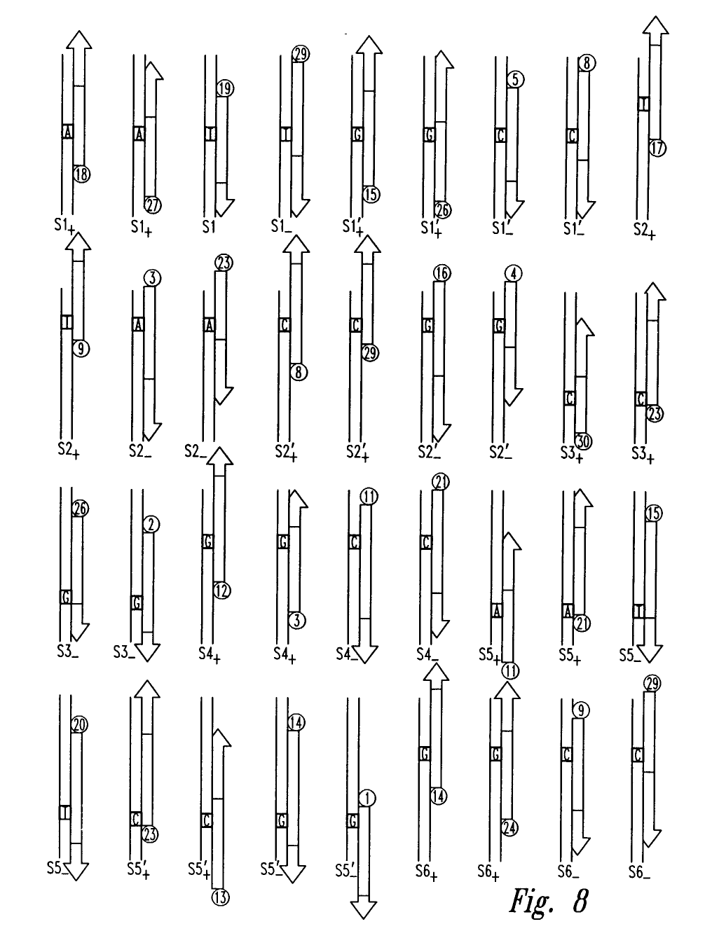

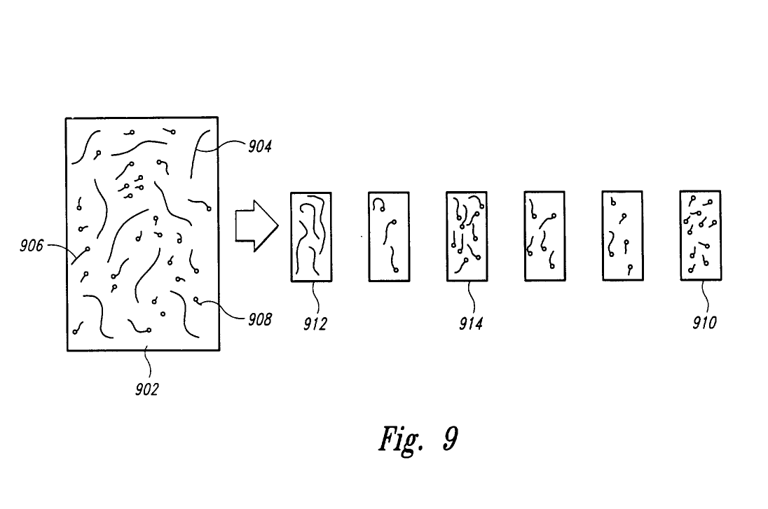

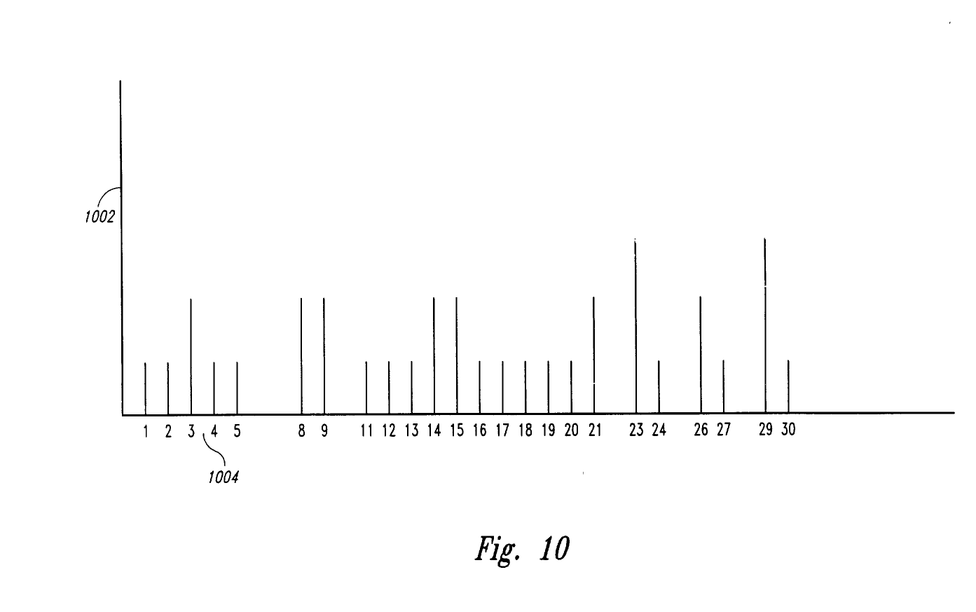

As discussed above, the method and system of the present invention concern mass-tagged primers and DNA sequences containing SNPs. In general, an SNP is bi-allelic or, in other words, comprises two different alleles that differ by a single base pair. Genotyping is thus a generalized technique for determining which of two possible alleles for each SNP of a set of SNPs are present in the genome of a given organism. Note that unlike in the simplified example described with reference to FIGS. 4-10, individuals may be homozygous or heterozygous with respect to a given SNP, so that both allelic sequences of an SNP sequence pair may be found in a DNA sample obtained from a single individual.

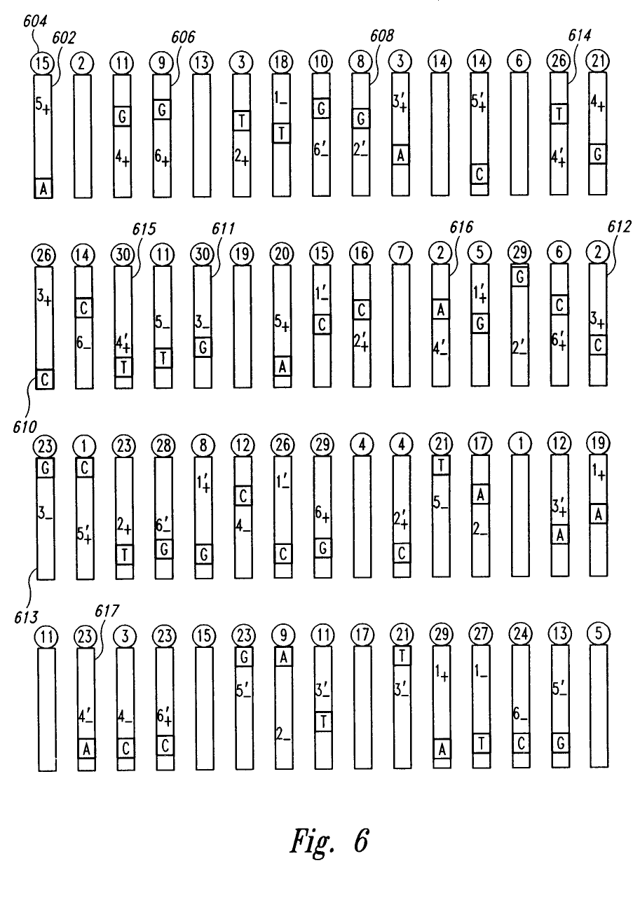

In the following, sets of oligonucleotide primers are assumed to comprise short, oligonucleotide primers having a common length. For example, the set of primers shown in FIG. 6 is a subset of the set of possible DNA oligonucleotides of length 6. Mathematically, this set of primers is described as a subset of the set of all possible DNA oligonucleotides having length 6, or six-mers, expressed as follows:

P⊂{A, C, G, T}⁶

where P is a set of primers used in a genotyping assay

The sequence of a given primer is denoted by the symbol “π,” where

π∈P

In the case of CMT-labeled oligonucleotide primers used in the CMT/PEA technique, the function z(π) returns the molecular weight, or mass, of the mass tag associated with sequence π.

Similarly, DNA-polymer sequences containing SNPs, such as SNP-containing chromosome fragments, or PCR amplicons of those SNP-containing chromosome fragments, are mathematically represented as follows:

S={A, C, G, T}*

Since the current technique is directed towards partitioning bi-allelic SNP-containing sequences, it is convenient to consider pairs of DNA-polymer sequences, where a pair of sequences q comprising the sequences s and t is denoted as:

q=(s,t)

where s∈S and t∈S

As discussed above, the partitioning techniques of the present invention are designed normally to partition sets of bi-allelic pairs of SNPs. An SNP comprises, in the case of bi-allelic SNPs, a sequence pair. A set, for the sake of SNP-based genotyping, is a set of bi-allelic SNPs, or sequence pairs, and is designated as:

Qᵢ

where i is an integer label.

The sequences within a set of bi-allelic SNPs can be expressed as follows:

seq(Q₀)={s:(s,t)=q for some t and some q∈Q₀}

The mass spectrum for a DNA-polymer sequence, such as a gene fragment or PCR amplicon of a gene fragment, is defined as follows:

σ_{P}(s)={z(π): π is WC complementary to a substring of s}

where π∈the set of primers P,

σ_{P}(s) is the mass spectrum resulting from CMT/PEA analysis of sequence s using the set of primers P, and

z(π) is a function that returns the molecular weight of the mass tag associated with sequence π.

There are numerous alternative SNP-based genotyping assays. One alternative is the mass-spectrometry polymerase extension assay (“MS/PEA”) in which oligonucleotide primers without mass-tag labels are employed, as in the above-described CMT/PEA technique. Certain of the unlabeled primers hybridize to complementary sequences, and are extended by DNA polymerase. The extended primers can be separated from non-extended primers, and the molecular weights of the extended primers determined by mass spectrometry. The number and molecular weights of the extended primers then serves to identify the SNPs present within an analyzed sample when the primers are selected to produce unique mass spectra for each possible combination of SNPs within each analyzed sample, just as the mass spectrum generated from cleaved mass tags serves as a signature for a particular set of SNPs in the CMT/PEA method. In the MS/PEA technique, the function z is defined to be:

z(π)=mass of π

and

σ_{P}(s)=(z(π): πis WC complementary to a substring of s}

A second, alternative technique is based on k-mer arrays. A k-mer array contains a large number of features, each of which contains a large number of oligonucleotide probe molecules of length k having identical sequences. In a k-mer SNP genotyping assay, the molecular array is exposed to a sample solution containing labeled PCR amplicons of DNA fragments. Those features to which DNA fragments hybridize are then identified, via a label-detection technique, such as detecting fluorescent emission from the features of the array. The sequence of the probes within the features to which labeled DNA fragments hybridize then represent a collection of complementary oligonucleotides of length k. When the probe sequences are properly chosen, the SNPs in the sample solution can be uniquely determined from the set of features, and corresponding probe sequences, to which the DNA fragments hybridize. For k-mer-array-based techniques, the function z is defined to be:

z(π)=π

and

σ_{P}(s)={z(π): πis WC complementary to a substring of s}

where π is a k-mer molecular array probe sequence of length k.

The partitioning methods described below can be used for CMT/PEA data, MS/PEA data, and k-mer array data, providing that the appropriate definitions for the functions z and σ_{P}, described above, are used for data generated by the corresponding technique. The partitioning methods can be applied to many other types of array data produced by an almost limitless number of array-based techniques.

Returning to the CMT/PEA example, the mass spectrum comprises the masses of all CMT labels bound to primers complementary in sequence to subsequences of the DNA-polymer sequence s. Further useful definitions include:

σ_{P}(q)=σ_{P}(s)∪σ_{P}(t)

σ_{P}(Q₀)=∪{σ_{P}(q): q∈Q₀}

σ_{P}(S₀)=∪{σ_{P}(s):s∈S₀}

where σ_{P}(q) is the mass spectrum of a pair of sequences q,

σ_{P}(Q₀) is the mass spectrum of a set of sequence pairs, or partition, Q₀, and

σ_{P}(S₀) is the spectrum of a set of sequences S₀

With the above definitions, and in view of the above discussion of the example CMT/PEA assay of the FIGS. 4-10, a fundamental assignability constraint can be defined for a partition Q₀. A set of sequence pairs Q₀ is assignable with respect to a set of primers P if:

for any member q=(s, t)∈Q₀ there is a mass z(s) such that z(s)∈σ_{P}(s), but z(s)∉σ_{P}(seq(Q₀)−s), and there is a mass z(t) such that z(t)∈σ_{P}(t), but z(t)∈σ_{P}(seq(Q₀)−t)

In other words, a set of sequence pairs is assignable with respect to a subset of primers P if there is a unique mass-spectrum peak, or CMT-label molecular weight, for each sequence of each pair of sequences in partition Q₀. The unique CMT-label molecular weights are referred to as the “unique peaks” of sequence pair q=(s,t) with respect to partition Q₀ and primer set P. The number of unique peaks for a sequence s with respect to a set of primers P is denoted:

N_{P}(s)

In the above-described mathematical model, a genotype with respect to a partition represents a selection of one or both sequences from each pair of sequences in the partition that occur in the chromosomal DNA isolated from an individual organism. A set of sequence pairs Q₀ is assignable with respect to primer set P if the genotype of an individual with respect to primer set P can be determined by a single CMT/PEA assay using the set of primers P. The computational goal of the present invention can therefore be stated as follows: given primer sets P₁, P₂, . . . Pᵣ and SNP sequence pairs q₁, q₂, . . . qₙ, find a partition of the SNP sequence pairs q₁, q₂, . . . qₙ into sequence-pair sets Q₁, Q₂, . . . Qₖ, each associated with a primer determined using the function “P,” where P(Qᵢ)∈{P₁, P₂, . . . Pᵣ}, such that, for all i, Qᵢ is assignable with respect to P(Qᵢ) and k is minimal. It should be noted that an organism normally contains paired chromosomes, and thus may be homozygous for one of two bi-allelic sequences of an SNP sequence pair, or may be heterozygous, having both sequences of an SNP pair, one from each chromosome.

### Partitioning Methods Represented In Mathematical Pseudocode

A first method for partitioning a set Q of sequence pairs into partitions Q₁, Q₂, . . . Qₖ that are assignable with respect to a single fixed set of primers “P,” i.e. r=1, is described, below, in pseudocode:

1 randomly order the pairs q₁ . . . qₙ, where qᵢ∈{qᵢ}ᵢ₌₁ⁿ

2 set Q₁=∅, k=1

3 for i=1 . . . n consider the pair qᵢ=(s,t)

4 find an index 1≦j≦k so that

5 (Qⱼ∪{qᵢ} is assignable with respect to P AND

6 σ_{P}(Qⱼ∪{qᵢ}) is minimal with respect to j)

7 if such a j is found,

8 put qᵢ into Qⱼ

9 else

10 set Qₖ₊₁=qᵢ, and k=k+1

11 end if

12 end for

The above method, called the “minimal partition heuristic” (“MPH”), begins, on line 1, by randomly ordering the bi-allelic pairs qᵢ. As an alternative to this first step, the pairs may be sorted in ascending order with respect to the number of unique peaks in the spectrum of the two sequences of the pair with respect to the set of primers P. Next, on line 2, an initial first partition Q₁ is created, with no members, and the local variable k is set to 1. Next, in the for-loop of lines 3-12, each bi-allelic sequence pair qᵢ in the set of sequences Q is placed into an existing partition or into a new partition created for the sequence pair. For each of the sequence pairs in the set of sequence pairs Q, lines 4-11 is executed during execution of the for-loop of lines 3-12. First, on line 4, an index j for an already created partition Qⱼ is found to which to add qᵢ if possible according to the conditional expressed on lines 5 and 6. A partition Qⱼ is selected as a candidate partition if the union of qᵢ, the set of sequences currently considered during the current iteration of the for-loop, and the partition Qⱼ is assignable with respect to the set of primers P. A final Qⱼ is selected from among the candidate partitions according to the criterion that the spectrum of the union of the final, selected partition Qⱼ and sequence qᵢ with respect to the set of primers P needs to be minimal with respect to all candidate Qⱼ, meaning that the spectrum of the final, selected partition Qⱼ, has the smallest number of peaks from among the spectra of the unions of qᵢ with any other candidates Qⱼ. If such an index j is found, as determined by MPH on line 7, then the currently considered sequence pair qᵢ is inserted into partition Q_{j.} Otherwise, on line 10, a new partition Qₖ₊₁ is created, with sequence pair qᵢ as its first member, and the index k is incremented.

In the case that several partitions, following the addition of sequence pair qᵢ, would have the same, minimal number of peaks with respect to primer set P, as determined on line 6, then an index j of the final, selected partition is chosen so that qᵢ is added to the partition Qⱼ that produces a spectrum with respect to the set of primers P having the largest number of characteristic masses. In the event that this secondary test also yields more than a single index j, then a single index j can be selected from among the candidate indices by one of many different techniques, including arbitrary selection based on random or pseudo-random numbers.

A second method for partitioning a set of bi-allelic sequence pairs Q={qᵢ}ᵢ₌₁ⁿ into several partitions Q₁, Q₂, . . . Qₖ is called the “maximal set heuristic” (“MSH”), and is provided below in a mathematical pseudocode implementation:

1 set Q₁=∅, k=1

2 while Q not empty

3 find a pair of sequences q=(s,t)ÅQ such that:

4 (Qₖ∪{q}) is assignable with respect to P AND

5 (Qₖ∪{q}) is minimal with respect to q)

6 if a q was found, then insert q into Qₖ and remove q from Q

7 else set Qₖ₊₁=∅and k=k+1

8 end while

The MSH routine starts, on line 1, with an initial partition Q₁ with no members and the partition index k equal to one. The while-loop of lines 2-8 iterates until the initial set of sequence pairs Q is empty or, in other words, until all sequence pairs have been assigned to partitions. During iteration of the while-loop, beginning on line 3, the MSH routine searches for a pair of sequences q as yet not assigned to a partition that can be added to the currently considered partition Qₖ to form a larger, but still assignable, partition and that, among all such candidate sequences q that can be added to the currently considered partition Qₖ, provides a larger partition Qₖ∪q with the smallest number of peaks in a mass spectrum resulting from a CMT/PEA assay, or other genotyping assay based on a final mass spectrum. If such a sequence pair q can be found, as determined on line 6, then that found sequence pair q is inserted into the currently considered partition Qₖ and removed from the set of unassigned sequence pairs Q. Otherwise, on line 7, a new partition Qₖ₊₁ is created with no members and the partition index k is incremented.

The above two methods can be modified in order to employ different sets of primers for each CMT/PEA or other genotyping assay. In other words, a different set of primers may be employed with the CMT/PEA technique for each partition. Thus, the modified MPH and MSH algorithms employ a set of primers Pᵢ. This set of primers can be generated by specifying the number of sets of primers to be used, r, and the depth of each set of primers dᵢ. The number of primers in a set of primers Pᵢ with CMT label of mass m≈dᵢ, when possible. In a first step of both the modified MPH and MSH methods, an initial correspondence between sets of primers Pᵢ and sequence pairs q is constructed, and each sequence pair q is additionally associated with an initial score. For a given sequence pair q and a given set of primers Pᵢ, the number of unique peaks for the sequence pair q with respect to the set of primers Pᵢ is defined to be:

Nᵢ(q)=min{Nᵢ(s),Nᵢ(t)}

where Nᵢ(s)=N_{P}_{ᵢ}(s)

In other words, the number of unique peaks for the sequence pair q is the minimum of the number of unique peaks for the two sequences that together compose the sequence pair. The initial score for a sequence q is then defined to be:

N(q)=maxᵢ(Nᵢ(q))

where Nᵢ(s) is defined, above, to be the number of unique peaks for a sequence s with respect to a set of primers Pᵢ. In other words, the initial score N(q) is the maximum number of unique peaks for the sequence pair q with respect to the sets of primers P₁, P₂, . . . Pᵣ, where r=number of different primer mixtures. This maximum number of unique peaks occurs upon application of some set of primers Pᵢ to the sequences of the sequence pair q in a CMT/PEA or analogous genotyping assay. The index i₀(q) for a given sequence q is defined to be the index of the set of primers that, when applied to the sequences of the sequence pair q, provides the initial score N(q), as discussed above. Finally, the partition initially assigned to a sequence pair q, P(q), is defined to be Pᵢ_{₀}_{(q)}.

The modified MPH method is provided below:

1 assign to each pair q₁ . . . qₙ, where qᵢ∈{qᵢ}ᵢ₌₁ⁿ=Q, primer mixtures P(q₁) . . . P(qₙ),

2 order the pairs qᵢ in ascending order with respect to their scores N(qᵢ)

3 set Q₁=Ø, P(Q₁)=P(q₁), k=1

4 for i=1 . . . n consider the pair qᵢ=(s,t)

5 find an index j where 1≦j≦k so that:

6 Qⱼ∪qᵢ is assignable with respect to P(Qⱼ) and

7 σ_{Pj}(Qⱼ∪qᵢ) is minimal for j with respect to the number of peaks

8 if an index j is found, then insert qᵢ into Qⱼ

9 else set Qₖ₊₁=qᵢ, P(Q₍ₖ₊₁₎)=P(q1), and k=k+1

10 end for

In the first step, on lines 1-2, each sequence pair q₁, q₂, . . . qₙ is associated with a set of primers P(q1), P(q₂), . . . P(qₙ), by the technique described above. The sequence pairs are sorted into ascending order with respect to their respective initial scores N(qᵢ). Next, on line 3, an initial partition Q₁ is created with no members and no associations with a set of primers, and a partition index k is set to 1. Then, in the for-loop of lines 4-9, a next sequence pair qᵢ is considered in each iteration. During each iteration, an index j is sought such that, if the currently considered sequence pair qᵢ is added to partition Qⱼ with respect to the set of primers P(Qⱼ) associated with partition Qⱼ, and the mass spectrum for the new partition formed by adding sequence qᵢ to partition Qⱼ is minimal for all possible candidate partitions Qⱼ. If an index j is found that meets the above-listed criteria, then sequence qᵢ is inserted into partition Qⱼ on line 10. Otherwise, a new partition Q₍ₖ₊₁₎ is created with the single member qᵢ and associated with the set of primers P(qᵢ) and the partition index k is incremented on line 11.

A modified MSH algorithm that employs multiple sets of primers is provided below:

1 assign to each pair q₁ . . . qₙ, where qᵢ∈{qᵢ}ᵢ₌₁ⁿ=Q, primer mixtures P(q₁) . . . P(qₙ),

2 ordering pairs qᵢ in ascending order with respect to their scores N(qᵢ)

3 set Q₁=Ø, P(Q₁)=P(q₁), k=1

4 while Q not empty

5 find a pair of sequences q=(s,t)∈Q so that:

7 Qₖ∪q is assignable with respect to P(Qₖ) and

8 σ_{P(q)}(Qₖ∪q) is minimal for all qÅQ with respect to the number of peaks

9 if a q is found then

10 insert q into Qₖ

11 remove q from Q

12 end if

13 else set Q₍ₖ₊₁₎=Ø, P(Q₍ₖ₊₁₎)=P(q1), and k=k+1

14 end while

In the first step, on lines 1-3, the modified MSH associates an initial score and an initial set of primers with each sequence pair q₁, q₂, . . . q, in the manner discussed above and employed as a first step in the modified MPH method. Next, on line 3, the modified MSH method creates an initial partition Q₁ with no members and sets the partition index k to 1. Next, the while-loop of lines 4-16 is executed until the set of unassigned sequence pairs Q is empty. On lines 5-9, during each iteration of the while-loop of lines 4-14, a sequence pair q is found so that it can be added to the currently considered partition Qₖ to produce a larger assignable partition Qₖ with respect to a set of primers associated with partition Qₖ, P(Qₖ), and so that the spectrum of the new partition has the smallest number of peaks of all sequence pairs q that can be added to partition Qₖ to produce a larger partition Qₖ assignable with respect to P(Qₖ). If such a sequence pair q is found, then the sequence pair q is inserted into partition Qₖ on line 10 and removed from Q on line 11. Otherwise, on line 14, a new partition Qₖ₊₁ is created with no members and the partition index k is incremented.

### Flow-Control Description of the MPH Method

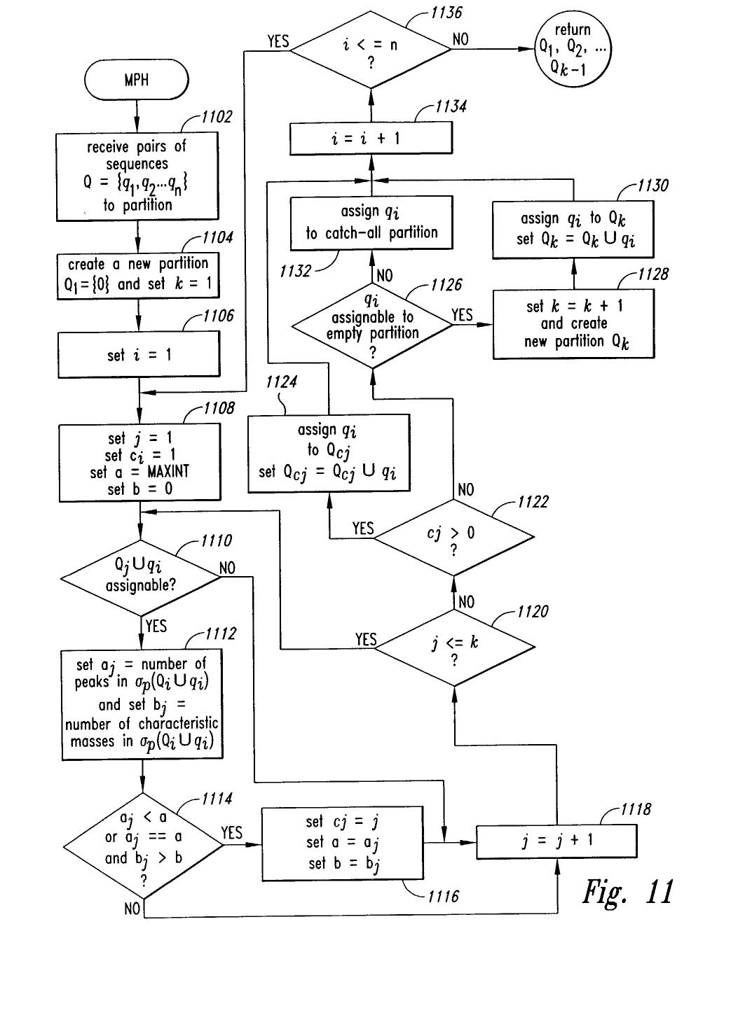

FIG. 11 is a flow-control diagram illustrating the MPH method described above in mathematical pseudocode. In step 1102, the MPH routine receives an initial set of pairs of sequences Q. In step 1104, the MPH routine creates a new empty partition Q₁ and sets the partition index variable “k” to 1. In step 1106, the MPH routine sets the for-loop control variable “i” to 1. In step 1108, the MPH routine sets an inner for-loop control variable “j” to 1, and local variables “cj,” “a,” and “b” to 0. Local variable “cj” saves the most favorable candidate partition for insertion of a sequence pair, local variable “a” stores the number of peaks in the mass spectrum that results in adding a sequence pair to the most favorable candidate partition, and local variable “b” stores the number of characteristic masses that would result in a mass spectrum of a partition formed by adding a candidate sequence pair to the most favorable candidate partition. Step 1106 is a first step of an outer for-loop and step 1108 is the first step of an inner for-loop.

The inner for-loop of steps 1108-1120, controlled by loop variable “j,” corresponds to the step, on lines 4-6 of the mathematical pseudocode for the MPH method, provided above, of finding an index j meeting the criteria of lines 5-6 of the mathematical pseudocode for the MPH method. In step 1110, the MPH routine determines whether the partition created by adding sequence pair “qᵢ” to the already created partition “Qⱼ” would be assignable with respect to the set of primers used in a CMT/PEA genotyping assay or other similar genotyping assay. If the partition “Qⱼ∪qᵢ” is not assignable, then the inner for-loop variable “j” is incremented, in step 1118, and, in step 1120, the MPH routine determines whether all currently created partitions have been considered, in the inner for-loop, as candidate partitions into which the sequence pair “qᵢ” can be included. If all current partitions have not been considered, control flows back to step 1110. If the partition “Qⱼ∪qᵢ” is assignable, as determined in step 1110, then the MPH routine sets local variable “n” to the number of peaks in the mass spectrum of partition “Qⱼ∪qᵢ” and sets local variable “c” to the number of characteristic masses in the spectrum of “Qⱼ∪qᵢ” in step 1112.

In step 1114, the MPH routine determines whether the partition “Qⱼ∪qᵢ” is minimal with respect to the number of peaks or, if this new partition and a previously considered partition are minimal, whether the partition “Qⱼ∪qᵢ” would provide the greatest number of characteristic masses of all partitions so far considered in the inner for-loop. If so, then the partition “Qⱼ∪qᵢ” becomes the best candidate partition so far considered in the inner for-loop, and local variables “cj,” “a,” and “b” are set to the values of local variables “j,” “n,” and “c,” respectively, in order to note that the partition “Qⱼ∪qᵢ” is a best candidate partition so far considered in the inner for-loop in step 1116. If the currently considered partition “Qⱼ∪qᵢ” is not the best candidate partition for adding sequence pair “q_{i,}” then control flows to step 1118. Thus, in the inner for-loop of steps 1108-1120, all currently created partitions are considered for addition of the sequence pair “qᵢ.”

Following completion of the inner for-loop, the MPH routine, in step 1123, determines whether or not a candidate partition from among the currently created partitions Q1, Q2, . . . Qₖ has been identified in the inner for-loop of steps 1108-1120. If so, then in step 1124, the MPH routine assigns sequence pair “qᵢ” to the best candidate partition “Q_{cj}” and control flows to step 1134. If a suitable candidate partition is not found in the inner for-loop, as detected in step 1122, then in step 1126, the MPH routine determines whether the sequence pair “qᵢ” is assignable to an empty partition. If so, then, in step 1128, the MPH routine increments the partition index “k” and creates the new partition “Qₖ.” Then, in step 1130, the MPH routine assigns “qᵢ” to the newly created partition “Qₖ.” If the sequence pair “qᵢ” is not assignable to an empty partition, then, in step 1132, the MPH routine assigns sequence pair “qᵢ” to a catch-all partition. In step 1134, the outer for-loop control variable “r” is incremented and the MPH routine then determines, in step 1136, whether there are more sequence pairs to consider in subsequent iterations of the outer for-loop. If so, then control flows back to step 1108. Otherwise, the MPH routine returns the partitions Q₁, Q₂, . . . Qₖ₋₁.

### C++-Like Implementaiton of the MPH Method

In this subsection, a C++-like implementation of the MPH method is provided as one example of how the mathematical pseudocode descriptions can be implemented in a common computer language. As with all software, there are essentially an unlimited number of ways to implement a particular method, with endless variations possible in data structures, flow control, modularization, and a host of other characteristics.

First, a class “oligo” is declared to represent a DNA-polymer sequence, such as a chromosome fragment or PCR amplicon of a chromosome fragment. Implementations of several member functions of the class “oligo” follow the class declaration:

| | |
|---|---|
| 1 | enum BASE {A, C, G, T}; |
| 2 | typedef unsigned_int32 SEQ_EL; |
| 3 | |
| 4 | |
| 5 | class oligo |
| 6 | { |
| 7 | private: |
| 8 | SEQ_EL sequence[maxSeqEl]; |
| 9 | int len; |
| 10 | |
| 11 | public: |
| 12 | inline int getLen( ) {return len; }; |
| 13 | inline SEQ_EL* getCode( ) {return sequence;}; |
| 14 | BASE getBase(int i); |
| 15 | void display(FILE *fptr); |
| 16 | void display( ); |
| 17 | void setSeq(char* seq); |
| 18 | oligo( ); |
| 19 | oligo(char* seq); |
| 20 | virtual ˜oligo( ); |
| 21 | }; |
| 22 | BASE oligo::getBase(int i) |
| 23 | { |
| 24 | unsigned char s; |
| 25 | int k; |
| 26 | |
| 27 | k = i/16; |
| 28 | i = i % 16 * 2; |
| 29 | s = (unsigned char) (sequence[k] >> i) & 0x3; |
| 30 | return (BASE) s; |
| 31 | } |
| 32 | |
| 33 | void oligo::setSeq(char* s) |
| 34 | { |
| 35 | int i = 0; |
| 36 | int j, m; |
| 37 | unsigned char nxt; |
| 38 | unsigned int k; |
| 39 | |
| 40 | j = 0; |
| 41 | k = 0; |
| 42 | m = 0; |
| 43 | while (*s != ‘\0’) |
| 44 | { |
| 45 | if(*s == ‘A’) nxt = 0; |
| 46 | else if (*s == ‘C’) nxt = 1; |
| 47 | else if (*s == ‘G’) nxt = 2; |
| 48 | else nxt = 3; |
| 49 | k = (nxt << (2 * m)) \|k; |
| 50 | s++; |
| 51 | i++; |
| 52 | m++; |
| 53 | if (m == 16) |
| 54 | { |
| 55 | sequence[j] = k; |
| 56 | m = 0; |
| 57 | j++; |
| 58 | k = 0; |
| 59 | } |
| 60 | } |
| 61 | if (j < maxSeqEl) sequence[j] = k; |
| 62 | len = i; |
| 63 | } |

The class “oligo” stores a DNA base sequence in the integer array “sequence,” declared on line 8. Each position of the sequence is represented by two bits, with the bases A, C, G, and T represented by bit patterns 00, 01, 10, and 11, respectively. This encoding is space efficient, and allows for a simple complementarity test based on bit wise logical operators. The length, in bases, of the DNA polymer represented by an instance of the class “oligo” is stored in the private member data “len,” declared on line 9. The class “oligo” includes member functions that return the length of the DNA-polymer sequence, a pointer to the sequence data stored in integer array “sequence,” and the identity of the base at a specified position within the sequence on lines 12-14, respectively. The class “oligo” also includes a member function to store a sequence specified as a character string into the integer array “sequence,” declared on line 17. The class “oligo” includes various additional member functions, two constructors, and a destructor.

The class “oligo” member function “getBase,” provided on lines 22-31, returns an indication of a nucleotide at a position within the DNA sequence specified by the argument “i.” This member function determines the two bits within the integer array “sequence” corresponding to the base at position “i” and returns an indication of the base on line 30. An implementation of the class “oligo” member function “setSeq,” which transforms an input character string “s” into a sequence of two-bit base codes that are stored, in order, into member data “sequence,” is provided on lines 33-63. In the while-loop of lines 43-60, setSeq traverses the character string, transforming characters indicating nucleotide bases into two-bit base designations and storing them within the integer array “sequence.”

Next, a declaration of the class “primer” and an implementation of a member function of the class “primer” are provided:

| | |
|---|---|
| 1 | typedef primer* Pptr; |
| 2 | unsigned char masses[65536]; |
| 3 | int primerLength; |
| 4 | |
| 5 | class primer |
| 6 | { |
| 7 | private: |
| 8 | unsigned int dex; |
| 9 | |
| 10 | public: |
| 11 | inline unsigned int getCode( ) {return dex;}; |
| 12 | inline void setCode(unsigned int d) {dex = d;}; |
| 13 | inline int getMass( ) {return masses[dex];}; |
| 14 | inline void setMass(int mass) {masses[dex] = mass;}; |
| 15 | BASE getBase(int i); |
| 16 | void display( ); |
| 17 | void display(FILE* fptr); |
| 18 | primer( ); |
| 19 | virtual ˜primer( ); |
| 20 | }; |
| 21 | BASE primer::getBase(int i) |
| 22 | { |
| 23 | unsigned char s; |
| 24 | int j; |
| 25 | |
| 26 | if (i >= primerLength) return X; |
| 27 | j = primerLength − i − 1; |
| 28 | s = (dex >> (2 * j)) & 0x3; |
| 29 | return (BASE) s; |
| 30 | } |

Next, the class “setOfPrimers” is declared, and several implementations of member functions provided:

| | |
|---|---|
| 1 | typedef primer* Pptr; |
| 2 | class setOfPrimers |
| 3 | { |
| 4 | private: |
| 5 | Pptr primers; |
| 6 | int num; |
| 7 | int added; |
| 8 | |
| 9 | public: |
| 10 | inline int getNum( ) {return num;}; |
| 11 | Pptr getPrimer(int i) {return &(primers[i];}; |
| 12 | int addPrimer(unsigned int dex, int mass); |
| 13 | setOfPrimers(int n); |
| 14 | virtual ˜setOfPrimers( ); |
| 15 | }; |
| 16 | |
| 17 | typedef setOfPrimers* SOPptr; |
| 18 | int setOfPrimers::addPrimer(unsigned int dex, int mass) |
| 19 | { |
| 20 | Pptrp; |
| 21 | |
| 22 | if (added < num) |
| 23 | { |
| 24 | p = &(primers[added]); |
| 25 | p->setCode(dex); |
| 26 | p->setMass(mass); |
| 27 | added++; |
| 28 | } |
| 29 | return added; |
| 30 | } |
| 31 | setOfPrimers::setOfPrimers(int n) |
| 32 | { |
| 33 | primers = new primer[n]; |
| 34 | added = 0; |
| 35 | num = n; |
| 36 | } |
| 37 | setOfPrimers::˜setOfPrimers( ) |
| 38 | { |
| 39 | delete [ ]primers; |
| 40 | } |

The class “setOfPrimers” represents a set of oligonucleotide primers used in a CMT/PEA assay. This class is quite straightforwardly declared and implemented. The class “setOfPrimers” includes member functions, declared on lines 10-14, to return the number of primers within the set, to returns a pointer to the ith primer of the set, to add a primer to the set, and to construct and deconstruct an instance of the class “setOfPrimers.”

Next, the class “setOfSetsofPrimers” is declared, and implementations of several member functions provided:

| | |
|---|---|
| 1 | class setofSetsofPrimers |
| 2 | { |
| 3 | private: |
| 4 | int numSets; |
| 5 | int nextD; |
| 6 | inline void initNextD(int i) {nextD = i;}; |
| 7 | SOPptr* sets; |
| 8 | inline unsigned int getNextDex( ) {return nextD++;}; |
| 9 | |
| 10 | public: |
| 11 | inline int getNum( ) {return numSets;}; |
| 12 | inline setOfPrimers* getSetOfPrimers(int i) {return sets[i];}; |
| 13 | setofSetsofPrimers(int numInPrimer, int depth, int nums); |
| 14 | virtual ˜setofSetsofPrimers( ); |
| 15 | }; |
| 16 | const int maxMass = 100; |
| 17 | const int minSet = 128; |
| 18 | |
| 19 | setofSetsofPrimers::setofSetsofPrimers(int numInPrimer, int depth, |
| | int nums) |
| 20 | { |
| 21 | int i, j, k, l, nA; |
| 22 | double total; |
| 23 | unsigned int perSet; |
| 24 | unsigned int numAtDepth; |
| 25 | unsigned int numAdd; |
| 26 | unsigned int newDex; |
| 27 | |
| 28 | numSets = nums & 0x3F; |
| 29 | if(numSets < 1) numSets = 1; |
| 30 | if(numInPrimer > 8) numInPrimer = 8; |
| 31 | if (numInPrimer < 4) numInPrimer = 4; |
| 32 | primerLength = numInPrimer; |
| 33 | total = pow(4, numInPrimer); |
| 34 | while (total < numSets * minSet) numSets−−; |
| 35 | perSet = total/numSets; |
| 36 | if (depth < 1) depth = 1; |
| 37 | while (perSet < depth * maxMass) depth−−; |
| 38 | numAtDepth = depth * maxMass; |
| 39 | numAdd = perSet − numAtDepth; |
| 40 | |
| 41 | sets = new SOPptr[numSets]; |
| 42 | nextD = 0; |
| 43 | for (i = 0; i < numSets; i++) |
| 44 | { |
| 45 | 1 = 0; |
| 46 | initNextD(i); |
| 47 | sets[i] = new setOfPrimers(perSet); |
| 48 | for (j = 0;j < depth; j++) |
| 49 | { |
| 50 | for (k = 0; k < maxMass; k++) |
| 51 | { |
| 52 | newDex = getNextDex( ); |
| 53 | sets[i]->getPrimer(1)->setCode(newDex); |
| 54 | sets[i]->getPrimer(1++)->setMass(k); |
| 55 | } |
| 56 | } |
| 57 | nA = numAdd; |
| 58 | k = 0; |
| 59 | while (nA) |
| 60 | { |
| 61 | newDex = getNextDex( ); |
| 62 | sets[i]->getPrimer(1)->setCode(newDex); |
| 63 | sets[i]->getPrimer(1++)->setMass(k); |
| 64 | nA−−; |
| 65 | k++; |
| 66 | if(k == maxMass) k = 0; |
| 67 | } |
| 68 | } |
| 69 | } |
| 70 | setofSetsofPrimers::˜setofSetsofPrimers( ) |
| 71 | { |
| 72 | int i; |
| 73 | |
| 74 | for (i = 0; i < numSets; i++) delete sets[i]; |
| 75 | delete [ ] sets; |
| 76 | } |

The class “setofSetsofPrimers” represents a collection of sets of primers that can be used, for example, in implementing the modified MPH and MSH methods, described above. In the current implementation, only the unmodified MPH method is implemented, so that only a single set of primers is used below. The class “setofSetsofPrimers” includes public member functions, declared on lines 34-37, that return the number of setsOfPrimer instances in the collection of setsOfPrimers, return a pointer to a particular setOfPrimer instance in the collection of primers, and that construct and destruct an instance of the class “setofSetsofPrimers.” The constructor for the class “setofSetsofPrimers” is implemented on lines 18-68, above. The implementation is straightforward. In the for-loop of lines 43-67, each set of primers is constructed. In the for-loop of lines 47-54 and the while-loop of lines 57-6, primers having successive indexes into the array “masses” are allocated for the set of primers currently under construction, with the mass labels assigned to the primers successively. Because there are fewer mass labels and primer sequences, each different mass label ends up being assigned to multiple primers within a set of primers. Note that the constructor for the class “setofSetsofPrimers” takes the following input arguments: (1) “numInPrimer,” the number of bases in each primer sequence; (2) “depth,” the desired numbers of primers in each primer set having any particular CMT-label mass; and (3) “nums,” the number of sets of primers to include in the collection of sets of primers represented in an instance of the class “setofSetsofPrimers.”

The class “tools,” declared below along with implementations of several member functions, provides certain basic functions used in implementation of the MPH method:

| | |
|---|---|
| 1 | class tools |
| 2 | { |
| 3 | private: |
| 4 | void shift(unsigned_int32* ptr); |
| 5 | public: |
| 6 | bool valid (char c); |
| 7 | bool valid(char* s); |
| 8 | bool valid(char* s, int len); |
| 9 | void shift(unsigned_int32* ptr); |
| 10 | bool complementary(oligo* o, primer* p); |
| 11 | tools( ); |
| 12 | virtual ˜tools( ); |
| 13 | }; |
| 14 | tools TOOLS; |
| 15 | void tools::shift(unsigned_int32* ptr) |
| 16 | { |
| 17 | int i; |
| 18 | unsigned_int32 carry; |
| 19 | |
| 20 | for (i = 0; i < maxSeqEl − 1; i++) |
| 21 | { |
| 22 | carry = (*(ptr + 1) & 0x3) << 30; |
| 23 | *ptr++ = (*ptr >> 2) \| carry; |
| 24 | } |
| 25 | *ptr = *ptr >>2; |
| 26 | } |
| 27 | bool tools::complementary(oligo* o, primer* p) |
| 28 | { |
| 29 | |
| 30 | int i; |
| 31 | unsigned_int32 j = p->getCode( ); |
| 32 | unsigned_int32* ptr = o->getCode( ); |
| 33 | unsigned_int32* ptrc; |
| 34 | unsigned_int32 Scode[maxSeqEl]; |
| 35 | unsigned_int32 mask; |
| 36 | int numShifts; |
| 37 | |
| 38 | ptrc = Scode; |
| 39 | for (i = 0; i < maxSeqEl; i++) Scode[i] = *ptr++; |
| 40 | |
| 41 | numShifts = o->getLen( ) − primerLength; |
| 42 | mask = pow(4, primerLength) − 1; |
| 43 | if (numShifts <= 0) return false; |
| 44 | j = j & mask; |
| 45 | while (numShifts > 0) |
| 46 | { |
| 47 | numShifts−−; |
| 48 | shift(ptrc); |
| 49 | if(((j & *ptrc) == 0) && (((j \| *ptrc) & mask) == mask)) |
| 50 | return true; |
| 51 | } |
| 52 | return false; |
| 53 | } |

The class “tools” includes member functions, declared on lines 4-6 above, that check a supplied character string for validity, essentially checking to make sure that the character string contains only the characters “A,” “C,” “G,” and “T.” These member functions are straightforward, and implementations are not provided. The class “tools” also provides the member function “complementary” which determines whether the primer pointed to by the supplied primer pointer “p” is complementary to any subsequences within the DNA-polymer sequence represented by an instance of the class “oligo” pointed to by oligo pointer “o.” The member function “complementary” uses the private member function “shift” for traversing the DNA-polymer sequence represented by the instance of the class “oligo” pointed to by pointer “o.” An implementation of the private member function “shift” is provided on lines 15-26, above. This member function receives a pointer to an integer array containing an encoded DNA-polymer sequence. It shifts the two-bit patterns within the sequence by one place. An implementation of the public member function “complementary” is provided on lines 27-52. This member function checks complementarity of the primer sequence to the DNA-polymer sequence along the length of the DNA-polymer sequence. This member function requires that at least one base of a DNA-polymer sequence extend past the 3′ end of the primer sequence to allow for extension of the primer. Traversal of the DNA-polymer sequence is carried out in the while-loop of lines 45-50. Note that the base pairing between up to four primer bases and four corresponding DNA-polymer sequence bases can be carried out in two bit-wise logical operations in line 49.

A declaration of the class “seqPair” is provided below:

| | |
|---|---|
| 1 | class seqPair |
| 2 | { |
| 3 | private: |
| 4 | oligo seq1; |
| 5 | oligo seq2; |
| 6 | int part; |
| 7 | |
| 8 | public: |
| 9 | inline void setPart(int p) {part = p;}; |
| 10 | inline int getPart( ) {return part;}; |
| 11 | inline void setSeqs(char* s1, char* s2) |

| | |
|---|---|
| 12 | {seq1.setSeq(s1); seq2.setSeq(s2);}; |

| | |
|---|---|
| 13 | inline void getSeqs(oligo** o1, oligo** o2) |

| | |
|---|---|
| 14 | {*o1 = &seq1; *o2 = &seq2;}; |

| | |
|---|---|
| 15 | seqPair( ); |
| 16 | virtual ˜seqPair( ); |
| 17 | }; |

An instance of the class “seqPair” represents a pair of DNA-polymer sequences that are stored in data members “seq1” and “seq2,” declared above on lines 3 and 4. The private member function “part” declared on line 5, stores an integer identifier of a partition to which the pair of DNA-polymer sequences represented by an instance of the class “seqPair” has been assigned. Member functions declared for class “seqPair” allow for setting and retrieving the integer partition identifier and setting and retrieving DNA-polymer sequences. An instance of the class “seqPair” corresponds to the contents of a sequence pair variable “q” used in the above mathematical pseudocode implementations of the MPH, MSH, and modified MPH and MSH methods.

Finally, a declaration of the class “partition” is provided, below, along with implementations of significant member functions:

| 1 | const int maxPartitions = 75; | |
|---|---|---|
| 2 | class partition | |
| 3 | { | |
| 4 | private: | |
| 5 | int numSequencePairs; | |
| 6 | seqPair seq_ps[100]; | |
| 7 | setofSetsofPrimers sosop; | |
| 8 | int numPartitions; | |
| 9 | int pMassCounts[maxPartitions][maxMass]; | |
| 10 | | |
| 11 | public: | |
| 12 | inline int getNumSequencePairs( ) | |
| 13 | | {return numSequencePairs;}; |
| 14 | inline void addSequencePair(char* s, char* t) | |
| 15 | | {seq_ps[numSequencePairs].setSeqs(s, t); |
| | | numSequencePairs++;}; |
| 16 | inline seqPair* getSequencePair(int i) | |
| | {return &(seq_ps[i]);}; | |
| 17 | void assign(unsigned int part, seqPair* sp, int* sPeaks, | |
| | int* tPeaks); | |
| 18 | inline void addPartition( ) {numPartitions++;}; | |
| 19 | void deletePartition( ) {numPartitions−−;}; | |
| 20 | bool assignable(unsigned int part, seqPair* sp, | |
| | setOfPrimers* sop, | |
| 21 | | int & numPeaks, int & singletons, int* sPeaks, |
| 22 | | int* tPeaks); |

| 23 | void greedyMinimalPartitionHeuristic( ); | |
|---|---|---|
| 24 | void printPartitions( ); | |
| 25 | partition(int numInPrimer, int depth, int numSets); | |
| 26 | virtual ˜partition( ); | |
| 27 | }; | |
| 28 | void partition::assign(unsigned int part, seqPair* sp, int* sPeaks, | |
| | int* tPeaks) | |
| 29 | { | |
| 30 | int i; | |
| 31 | | |
| 32 | sp->setPart(part); | |
| 33 | for (i = 0; i < maxMass; i++) | |
| 34 | { | |
| 35 | pMassCounts[part][i] += sPeaks[i] + tPeaks[i]; | |
| 36 | } | |
| 37 | } | |
| 38 | bool partition::assignable(unsigned int part, seqPair* sp, | |
| | setOfPrimers* sop, | |
| 39 | | int & numPeaks, int & singletons, int* sPeaks, |
| | | int* tPeaks) |

| 40 | { | |
|---|---|---|
| 41 | int i; | |
| 42 | primer* p; | |
| 43 | oligo* s; | |
| 44 | oligo* t; | |
| 45 | bool uniqueS = false, uniqueT = false; | |
| 46 | int j; | |
| 47 | | |
| 48 | sp->getSeqs(&s, &t); | |
| 49 | for (i = 0; i < sop->getNum( ); i++) | |
| 50 | { | |
| 51 | p = sop->getPrimer(i); | |
| 52 | j = p->getMass( ); | |
| 53 | if (TOOLS.complementary(s, p)) | |
| 54 | sPeaks[j] = 1; | |
| 55 | if(TOOLS.complementary(t, p)) | |
| 56 | tPeaks[j] = 1; | |
| 57 | } | |
| 58 | numPeaks = 0; | |
| 59 | singletons = 0; | |
| 60 | for (i = 0; i < maxMass; i++) | |
| 61 | { | |
| 62 | j = sPeaks[i] + tPeaks[i] + pMassCounts[part][i]; | |
| 63 | if (j > 0) numPeaks++; | |
| 64 | if (j == 1) | |
| 65 | { | |
| 66 | if (sPeaks[i] == | |
| | 1 && tPeaks[i] == 0 && pMassCounts[part][i] == 0) | |
| 67 | uniqueS = true; | |
| 68 | if(sPeaks[i] == 0 && tPeaks[i] == | |
| | 1 && pMassCounts[part][i] == 0) | |
| 69 | uniqueT = true; | |
| 70 | singletons++; | |
| 71 | } | |
| 72 | } | |
| 73 | return uniqueS && uniqueT; | |
| 74 | } | |
| 75 | void partition::greedyMinimalPartitionHeuristic( ) | |
| 76 | { | |
| 77 | int i, j, k; | |
| 78 | int sPeaks[maxMass]; | |
| 79 | int tPeaks[maxMass]; | |
| 80 | int s1Peaks[maxMass]; | |
| 81 | int t1Peaks[maxMass]; | |
| 82 | setOfPrimers* sop; | |
| 83 | seqPair* sp; | |
| 84 | int numPks; | |
| 85 | int singles; | |
| 86 | int c_numPks, c_singles, c_Part; | |
| 87 | | |
| 88 | | |
| 89 | sop = sosop.getSetOfPrimers(0); | |
| 90 | | |
| 91 | addPartition( ); | |
| 92 | for (i = 0; i < getNumSequencePairs( ); i++) | |
| 93 | { | |
| 94 | c_numPks = 100000; | |
| 95 | c_singles = 0; | |
| 96 | c_Part = −1; | |
| 97 | sp = getSequencePair(i); | |
| 98 | for (j = 0; j < numPartitions; j++) | |
| 99 | { | |
| 100 | for (k = 0; k < maxMass; k++) | |
| 101 | { | |
| 102 | sPeaks[k] = 0; | |
| 103 | tPeaks[k] = 0; | |
| 104 | } | |
| 105 | if(assignable(j, sp, sop, numPks, singles, sPeaks, tPeaks)) | |
| 106 | { | |
| 107 | if ((numPks < c_numPks) ∥ | |
| 108 | | (numPks == c_numPks && singles > c_singles)) |
| 109 | { | |
| 110 | | c_Part = j; |
| 111 | | c_singles = singles; |
| 112 | | c_numPks = numPks; |
| 113 | | for (k = 0; k < maxMass; k++) |
| 114 | | { |
| 115 | | s1Peaks[k] = sPeaks[k]; |
| 116 | | t1Peaks[k] = tPeaks[k]; |
| 117 | | } |

| | |
|---|---|
| 118 | } |
| 119 | } |
| 120 | } |
| 121 | if (c_Part >= 0) assign(c_Part, sp, s1Peaks, t1Peaks); |
| 122 | else |
| 123 | { |
| 124 | addPartition( ); |
| 125 | j = numPartitions − 1; |
| 126 | for (k = 0; k < maxMass; k++) |
| 127 | { |
| 128 | sPeaks[k] = 0; |
| 129 | tPeaks[k] = 0; |
| 130 | } |
| 131 | if (assignable(j, sp, sop, numPks, singles, sPeaks, |
| | tPeaks)) |
| 132 | assign(j, sp, sPeaks, tPeaks); |
| 133 | else |
| 134 | { |
| 135 | sp->setPart(catchAll); |
| 136 | deletePartition( ); |
| 137 | } |
| 138 | } |
| 139 | } |
| 140 | printPartitions( ); |
| 141 | } |
| 142 | partition::partition(int numInPrimer, int depth, int numSets) |
| 143 | :sosop (numInPrimer, depth, numSets) |
| 144 | { |
| 145 | int i, j; |
| 146 | |
| 147 | numSequencePairs = 0; |
| 148 | numPartitions = 0; |
| 149 | |
| 150 | for (i = 0; i < maxPartitions; i++) |
| 151 | { |
| 152 | for (j = 0; j < maxMass; j++) |
| 153 | { |
| 154 | pMassCounts[i][j] = 0; |
| 155 | } |
| 156 | } |
| 157 | } |

The class “partition” includes the following private data members: (1) “numSequencePairs,” the number of sequence pairs, or bi-allelic SNPs, to be partitioned; (2) “seq_ps,” an array of seqPair instances for storing sequence pairs; (3) “sosop,” a setOfSetsOfPrimers instance containing a set of primers that are applied to the sequence pairs during the SNP/PEA genotyping assay; (4) “numPartitions,” the number of sequence-pair partitions generated by application of a partitioning method, such as the MPH method described above; and (5) “pMassCounts,” an accumulator used for keeping track of the number of peaks in a mass spectrum generated from the sequence pairs within currently active partitions. The class “partition” includes the follows member functions: (1) “getNumSequencePairs,” declared above on line 12, which returns the number of sequence pairs that are to be partitioned by a partitioning method, such as the MPH method; (2) “addSequencePair,” declared above on line 14, which adds a pair of sequences designated by character-string representations pointed to by supplied pointers “s” and “t” to the set of sequence pairs to be partitioned; (3) “getSequencePair,” declared above on line 16, which returns a specified pair of sequences within the total collection of sequence pairs to be partitioned; (4) “assign,” declared above on line 17, which assigns a sequence pair to a specified partition; (5) “addPartition,” declared above on line 18, which creates a new empty partition; (6) “deletePartition,” declared above on line 19, which removes a partition; (7) “assignable,” declared above on lines 20-22, which determines whether the partition resulting from adding a specified sequence pair to a specified partition is assignable with regard to a specified set of primers; (8) “greedyMinimalPartitionHeuristic,” declared above on line 23, which implements the unmodified MPH method; and (9) a print routine, constructor, and destructor, declared above on lines 24-26. There are straightforward relationships between the private data members and public member functions of the class “partition” and the variables and functions used in the mathematical pseudocode implementation of the unmodified MPH method, described above. The initial set of sequence pairs Q in the mathematical pseudocode corresponds to the collection of sequence pairs stored in the array “seq_ps.” The partitions Q₁, Q₂, . . . Qₖ are designated by the integer partition labels stored in the data member “part” within the seqPair instances stored within the array “seq_ps.” The partition index k corresponds to the data member “numPartitions” in the class “partition.” The pairs of sequences q₁, q₂, . . . qₙ are instances of the class “seqPair” stored in the array “seq_ps.” The public member function “assignable” in the class “partitions” implements the criteria in the mathematical pseudocode that need to be met for a partition index j in order for the currently considered sequence pair qᵢ to be added to partition Qⱼ. The public member function “assign” corresponds to the operation of inserting a sequence pair qᵢ into a partition Qⱼ. The public member function “addPartition” corresponds to creation of a new partition Qₖ₊₁.

An implementation of the public member function “assign” is provided, above, on lines 28-37. A sequence pair is assigned to a partition by calling the seqPair member function “setPart” on line 32 and then accumulating any mass-spectrum peaks for the two sequences into the private data member “pMassCounts” on lines 33-36.

An implementation of the public member function “assignable” is provided on lines 38-74. The member function “assignable” first, in the for-loop of lines 49-57, accumulates into local variables “speaks” and “tPeaks” the masses of any primers in the set of primers references by argument “sop” that are complementary to either of the sequences in the sequence pair referenced by the argument “sop.” Then, in the for-loop of lines 60-72, member function “assignable” determines whether there is at least one mass-spectrum peak that is unique for each sequence of a sequence pair with respect to all other sequence pairs currently in the partition identified in argument “part” with respect to the set of primers referenced by argument “sop.” In the for-loop, assignable also counts the total number of spectral peaks in the mass spectrum for the partition that would result from adding the sequence pair referenced by argument “sp” to the partition indicated by argument “part” as well as the number of single peaks or, in other words, peaks resulting from a labeled primer complementary to only one sequence within the partition.

An implementation of the public member function “greedyMinimalPartitionHeuristic” is provided on lines 75-141, above. This member function corresponds to the mathematical pseudocode implementation of the unmodified MPH method, described above in a previous subsection. On line 89, a reference “sop” to a single set of primers stored within data member “sosop” is returned via a call to the member function “getSetOfPrimers.” On line 91, an initial empty partition is created. The for-loop of lines 92-139 implements the single for-loop within the mathematical pseudocode implementation of the unmodified MPH method. This for-loop iterates over all the sequence pairs to be partitioned. For each sequence pair, the for-loop of lines 98-120 is executed to identify an existing partition to which the currently considered sequence pair may be assigned to create a larger assignable partition and which is minimal among all candidate assignable partitions. If the partition resulting from adding the currently considered sequence pair to the currently considered partition “j” is assignable, as determined on line 105, and if the mass spectrum resulting from the partition formed by adding the currently considered sequence pair to the currently considered partition “j” has less peaks than any other partition so far considered, as detected on line 107, then the currently considered partition becomes the most promising candidate to which to add the currently considered sequence pair by setting local variable “c_Part” to “j” on line 110. If a suitable currently existing partition was identified in the inner for-loop of lines 98-120, as determined on line 121, then the currently considered sequence pair is added to that partition via a call to the member function “assign” on line 121. Otherwise, on lines 124-127, a new partition is created and, if the sequence pair is assignable, as detected on line 131, the currently considered sequence is assigned to the new partition on line 132. Otherwise, the sequence pair is unassignable with respect to the set of primers referenced by pointer “sop,” and the sequence pair is added to a special catch-all partition on line 135.

A truncated main routine is provided, below, to indicate how the C++-like implementation of the MPH method is employed:

1 main(int argc, char*argv[ ])
2 {
3 partition p(6, 41, 1);
4 p.addSequencePair
5 (“AGTCTTCAGCTGTGGGTACGTCGGGAATCCGTTGTGCTTTT”,
6 “AGTCTTCAGCTGTGGGTACATCGGGAATCCGTTGTGCTTTT”);
7 p.greedyMinimalPartitionHeuristic( );
8 }

Note that a number of calls to the member function “addSequencePair” would normally be included prior to calling member function “greedyMinimalPartitionHeuristic” on line 7.

Primer sequences are stored within an unassigned integer “dex,” declared as number data on line 8. The encoding is similar to that of encoding of the sequences in the class “oligo,” described above, except that, while the DNA-polymer sequence of an instance of the class “oligo” is stored in 5′ to 3′ order, the base sequence of a primer is stored in 3′ to 5′ order. The member data “dex” also serves as an index into the array “masses,” declared above on line 2, that contains the CMT labels for the primers. The member functions declared for the class “primer” on lines 11-19 are similar to those declared for class “oligo.” An implementation of the member function “getBase,” similar to the identically named member function for class “oligo,” is provided above on lines 21-30.

Although the present invention has been described in terms of a particular embodiment, it is not intended that the invention be limited to this embodiment. Modifications within the spirit of the invention will be apparent to those skilled in the art. For example, an almost limitless number of implementations of the partitioning methods described above can be obtained by varying the modularization, flow control, data structures, programming language, and other characteristics of the above-described implementations. While the above-described implementations focused on bi-allelic, SNP-containing sequence pairs, the methods are easily and straightforwardly extended to include higher-order SNPs with three and four alleles, as well as polymorphisms including more than a single base pair substitution within a small enough region to be encompassed by a small, oligonucleotide primer. The above-described implementations deal only with sequences of unmodified DNA bases, but can be easily and straightforwardly extended for partitioning nucleotide polymers including modified bases and other types of bio-polymers that associate through sequence-based interactions.

A variant of CMT comprises a ligation step that replaces the polymerase extension step. Thus, using 6-mer primer mixtures, products of length 12, 18, 24, etc. are generated. HPLC separation can serve to physically separate these products from the non-ligated 6-mers. The resulting mass spectra, assuming this assay was performed, can be used as the input to the multiplexing procedure described in this invention.

The foregoing description, for purposes of explanation, used specific nomenclature to provide a thorough understanding of the invention. However, it will be apparent to one skilled in the art that the specific details are not required in order to practice the invention. The foregoing descriptions of specific embodiments of the present invention are presented for purpose of illustration and description. They are not intended to be exhaustive or to limit the invention to the precise forms disclosed. Obviously many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalents:

1. A method for partitioning a set of sequence groups into sequence-group partitions with respect to association with a set of subsequences, the individual associations between each sequence in a sequence-group partition and each subsequence in the set of subsequences computable and measurable as a set of values, a sequence-group partition assignable with respect to the set of subsequences when individual associations between the sequences of the sequence-group partition and the set of subsequences are uniquely determined by the measurable set of values, the method comprising:
creating a first empty partition;
considering each sequence group in the set of sequence groups, for each considered sequence group attempting to select a candidate sequence-group partition from among existing sequence-group partitions to which to add the considered sequence group to form a larger sequence-group partition assignable with respect to the set of subsequences,
when a candidate sequence-group partition is selected, adding the considered sequence group to the candidate sequence-group partition, and
when a candidate sequence-group partition is not selected, creating a new sequence-group partition and adding the considered sequence group to the new sequence-group partition.

2. The method of claim 1 wherein considering each sequence group in the set of sequence groups, for each considered sequence group attempting to select a candidate sequence-group partition from among existing sequence-group partitions to which to add the considered sequence group to form a larger sequence-group partition assignable with respect to the set of subsequences further comprises:
selecting sequence-group partitions that, when the considered sequence group is added to the sequence-group partitions, result in larger sequence-group partitions assignable with respect to the set of subsequences; and
selecting as a candidate sequence-group partition from among the selected existing sequence-group partitions a sequence-group partition that, when the considered sequence group is added to the sequence-group partition, produces a minimal set of computed values.

3. The method of claim 2 wherein, when more than one candidate sequence-group partition is selected, choosing as a final selected candidate sequence-group partition a candidate sequence-group partition producing a largest number of computed values among the selected candidate sequence-group partitions.

4. The method of claim 1 wherein the sequence-groups are nucleotide sequences, each sequence group representing polymorphic forms of a DNA fragment, and the set of subsequences are nucleotide sequences, selected to be complementary to one or more of the DNA fragments.

5. A set of computer instructions for carrying out the method of claim 1 encoded by one of:
storing the computer instructions in a machine readable medium;
transmitting the computer instructions over an electronic communications medium; and
printing the computer instructions in a human readable medium.

6. A computer system including a set of computer instructions for carrying out the method of claim 1 that:
partitions a set of sequence groups into sequence-group partitions; and
stores a representation of the sequence-group partitions in a computer readable medium.

7. A set of sequence-group partitions produced by the method of claim 1 encoded by:
storing representations of the sequence-group partitions in a machine readable medium;
transmitting representations of the sequence-group partitions over an electronic communications medium; and
printing representations of the sequence-group partitions in a human readable medium.

8. A method for partitioning a set of sequence groups into sequence-group partitions with respect to association with a set of subsequences, the individual associations between each sequence in a sequence-group partition and each subsequence in the set of subsequences computable and measurable as a set of values, a sequence-group partition assignable with respect to the set of subsequences when individual associations between the sequences of the sequence-group partition and the set of subsequences are uniquely determined by the measurable set of values, the method comprising:
creating an empty, sequence-group current partition;
while the set of sequence groups is not empty,
attempting to select a candidate group of sequences from the set of sequence groups that can be added to the current sequence-group partition to create a larger sequence-group partition assignable with respect to the set of subsequences,
when a candidate sequence group is selected, adding the candidate sequence group to the current sequence-group partition and removing the candidate sequence group from the set of sequence groups; and
when a candidate sequence group is not selected, creating a new, empty current sequence-group partition.

9. The method of claim 8 wherein attempting to select a candidate group of sequences from the set of sequence groups that can be added to the current sequence-group partition to create a larger sequence-group partition assignable with respect to the set of subsequences further comprises:
selecting, as provisional candidate sequence groups from the set of sequence groups, sequence groups that can be added to the current sequence-group partition to create a larger sequence-group partition assignable with respect to the set of subsequences; and
selecting as a candidate sequence group from among the selected provisional candidate sequence groups a sequence group that, when added to the current sequence-group partition, produces a minimal set of computed values.

10. The method of claim 8 wherein the sequence-groups are nucleotide sequences, each sequence group representing polymorphic forms of a DNA fragment, and the set of subsequences are nucleotide sequences, selected to be complementary to one or more of the DNA fragments.

11. A set of computer instructions for carrying out the method of claim 8 encoded by one of:
storing the computer instructions in a machine readable medium;
transmitting the computer instructions over an electronic communications medium; and
printing the computer instructions in a human readable medium.

12. A computer system including a set of computer instructions for carrying out the method of claim 8 that:
partitions a set of sequence groups into sequence-group partitions; and
stores a representation of the sequence-group partitions in a computer readable medium.

13. A set of sequence-group partitions produced by the method of claim 8 encoded by:
storing representations of the sequence-group partitions in a machine readable medium;
transmitting representations of the sequence-group partitions over an electronic communications medium; and
printing representations of the sequence-group partitions in a human readable medium.

14. A method for partitioning a set of sequence groups into sequence-group partitions with respect to association with a set of subsequence groups, the individual associations between each sequence in a sequence-group partition and each subsequence in a subsequence group computable and measurable as a set of values, a sequence-group partition assignable with respect to a subsequence group when individual associations between the sequences of the sequence-group partition and subsequences of the subsequence group are uniquely determined by the measurable set of values, the method comprising:
assigning to each sequence group in the set of sequence groups a subsequence group from the set of subsequence groups;
creating a first empty sequence-group partition;
considering each sequence group in the set of sequence groups, for each considered sequence group attempting to select a candidate sequence-group partition from among existing sequence-group partitions to which to add the considered sequence group to form a larger sequence-group partitions assignable with respect to the subsequence group associated with the candidate sequence-group partition,
when a candidate sequence-group partition is selected, adding the considered sequence group to the candidate sequence-group partition, and
when a candidate sequence-group partition is not selected, creating a new sequence-group partition, adding the considered sequence group to the new sequence-group partition, and associating with the sequence-group partition the subsequence group assigned to the considered sequence group.

15. The method of claim 14 wherein considering each sequence group in the set of sequence groups, for each considered sequence group attempting to select a candidate sequence-group partition from among existing sequence-group partitions to which to add the considered sequence group to form a larger sequence-group partitions assignable with respect to the subsequence group associated with the candidate sequence-group partition further comprises:
selecting existing sequence-group partitions that, when the considered sequence group is added to the selected sequence-group partitions, result in larger sequence-group partitions assignable with respect to the subsequence group associated with the selected sequence-group partitions; and
selecting as a candidate sequence-group partition from among the selected sequence-group partitions a sequence-group partition that, when the considered sequence group is added to the sequence-group partition, produces a minimal number of computed values.

16. The method of claim 15 wherein, when more than one candidate sequence-group partitions are selected, choosing as a final, selected candidate sequence-group partition a selected sequence-group partition producing the largest number of computable values from among the selected candidate partitions.

17. The method of claim 14 wherein the sequence-groups are nucleotide sequences, each sequence group representing polymorphic forms of a DNA fragment, and the set of subsequence groups are nucleotide sequences, selected to be complementary to one or more of the DNA fragments.

18. A set of computer instructions for carrying out the method of claim 14 encoded by one of:
storing the computer instructions in a machine readable medium;
transmitting the computer instructions over an electronic communications medium; and
printing the computer instructions in a human readable medium.

19. A computer system including a set of computer instructions for carrying out the method of claim 14 that:
partitions a set of groups sequences into sequence-group partitions; and
stores a representation of the sequence-group partitions in a computer readable medium.

20. A set of sequence-group partitions produced by the method of claim 14 encoded by:
storing representations of the sequence-group partitions in a machine readable medium;
transmitting representations of the sequence-group partitions over an electronic communications medium; and
printing representations of the sequence-group partitions in a human readable medium.

21. A method for partitioning a set of sequence groups into sequence-group partitions with respect to association with a set of subsequence groups, the individual associations between each sequence in a sequence-group partition and each subsequence in a subsequence group computable and measurable as a set of values, a sequence-group partition assignable with respect to a subsequence group when individual associations between the sequences of the sequence-group partition and subsequences of the subsequence group are uniquely determined by the measurable set of values, the method comprising:
assigning to each sequence-group in the set of groups sequences a subsequence group from the set of subsequence groups;
creating an empty, current sequence-group partition;
while the set of sequence groups is not empty,
attempting to select a candidate sequence group from the set of sequence groups that can be added to the current sequence-group partition to create a larger sequence-group partition assignable with respect to the subsequence group assigned to the current sequence-group partition,
when a candidate sequence group is selected, adding the candidate sequence group to the current sequence-group partition, removing the candidate sequence group from the set of sequence groups; and
when a candidate group of sequences is not selected, creating a new, empty current sequence-group partition.

22. The method of claim 21 wherein attempting to select a candidate sequence group from the set of sequence groups that can be added to the current sequence-group partition to create a larger sequence-group partition assignable with respect to the subsequence group assigned to the current sequence-group partition further comprises:
selecting provisional candidate sequence groups from the set of sequence groups that can be added to the current sequence-group partition to create a larger sequence-group partition assignable with respect to a subsequence group assigned to the current sequence-group partition; and
selecting as a candidate sequence group from among the selected provisional candidate sequence groups a sequence group that, when added to the current sequence-group partition, produces a minimal number of computable values.

23. The method of claim 21 wherein the sequence-groups are nucleotide sequences, each sequence group representing polymorphic forms of a DNA fragment, and the set of subsequence groups are nucleotide sequences, selected to be complementary to one or more of the DNA fragments.

24. A set of computer instructions for carrying out the method of claim 21 encoded by one of:
storing the computer instructions in a machine readable medium;
transmitting the computer instructions over an electronic communications medium; and
printing the computer instructions in a human readable medium.

25. A computer system including a set of computer instructions for carrying out the method of claim 21 that:
partitions a set of sequence groups into sequence-group partitions; and
stores a representation of the sequence-group partitions in a computer readable medium.

26. A set of sequence-group partitions produced by the method of claim 21 encoded by:
storing representations of the sequence-group partitions in a machine readable medium;
transmitting representations of the sequence-group partitions over an electronic communications medium; and
printing representations of the sequence-group partitions in a human readable medium.
